SEQUENCE LISTING (21 sequences)

SEQ ID NO: 1 - DNA, 408 bp, synthetic construct
  source 1..408: mol_type = other DNA
  misc_feature 1..408: note = Optimized human Gal1
atggcttgtggtcttgttgctagtaatcttaatttgaaacctggtgaatgtttgcgagttcgtggtgaagttgctcctgatgctaaatcatttgttcttaatcttggtaaagattcaaataatttgtgtcttcattttaatccacgttttaatgctcatggagatgcaaatacaattgtttgtaattcaaaagatggtggagcttggggtactgaacaaagagaagcagtatttccatttcaacctggatctgttgctgaagtatgtattacatttgatcaagcaaatcttactgtaaaacttccagatggttatgaatttaaatttcctaatcgacttaatttggaagctattaattatatggctgctgatggagattttaaaattaaatgtgttgcttttgattaa

SEQ ID NO: 2 - DNA, 422 bp, synthetic construct
  misc_feature 1..422: note = SuperGal1 with optimized sequence and including secretion peptide usp45
  source 1..422: mol_type = other DNA
ccatggcttcaggtcttgttgctagtaatcttaatttgaaacctggtgaatcattgcgagttcgtggtgaagttgctcctgatgctaaatcatttgttcttaatcttggtaaagattcaaataatttgtgtcttcattttaatccacgttttaatgctaatggagatgcaaatacaattgtttgtaattcaaaagatggtggagcttggggtactgaacaaagagaagcagtatttccatttcaacctggatctgttgctgaagtatgtattacatttgatcaagcaaatcttactgtaaaacttccagatggttatgaatttaaatttcctaatcgacttaatttggaagctattaattatatggctgctgatggagattttaaaattaaatgtgttgcttttgattaaggtaccaagctt

SEQ ID NO: 3 - DNA, 422 bp, synthetic construct
  misc_feature 1..422: note = SuperGal2 with optimized sequence and including secretion peptide usp45
  source 1..422: mol_type = other DNA
ccatggcttcaggtcttgttgctagtaatcttaatttgaaacctggtgaatcattgcgagttcgtggtgaagttgctcctgatgctaaatcatttgttcttaatcttggtaaagattcaaataatttgtgtcttcattttaatccacgttttaatgcttatggagatgcaaatacaattgtttgtaattcaaaagatggtggagcttggggtactgaacaaagagaagcagtatttccatttcaacctggatctgttgctgaagtatgtattacatttgatcaagcaaatcttactgtaaaacttccagatggttatgaatttaaatttcctaatcgacttaatttggaagctattaattatatggctgctgatggagattttaaaattaaatgtgttgcttttgattaaggtaccaagctt

SEQ ID NO: 4 - DNA, 422 bp, synthetic construct
  misc_feature 1..422: note = SuperGal3 with optimized sequence and including secretion peptide usp45
  source 1..422: mol_type = other DNA
ccatggcttcaggtcttgttgctagtaatcttaatttgaaacctggtgaatgtttgcgagttcgtggtgaagttgctcctgatgctaaatcatttgttcttaatcttggtaaagattcaaataatttgtgtcttcattttaatccacgttttaatgctaatggagatgcaaatacaattgtttgtaattcaaaagatggtggagcttggggtactgaacaaagagaagcagtatttccatttcaacctggatctgttgctgaagtatcaattacatttgatcaagcaaatcttactgtaaaacttccagatggttatgaatttaaatttcctaatcgacttaatttggaagctattaattatatggctgctgatggagattttaaaattaaatgtgttgcttttgattaaggtaccaagctt

SEQ ID NO: 5 - DNA, 422 bp, synthetic construct
  misc_feature 1..422: note = SuperGal4 with optimized sequence and including secretion peptide usp45
  source 1..422: mol_type = other DNA
ccatggcttcaggtcttgttgctagtaatcttaatttgaaacctggtgaatgtttgcgagttcgtggtgaagttgctcctgatgctaaatcatttgttcttaatcttggtaaagattcaaataatttgtgtcttcattttaatccacgttttaatgcttatggagatgcaaatacaattgtttgtaattcaaaagatggtggagcttggggtactgaacaaagagaagcagtatttccatttcaacctggatctgttgctgaagtatcaattacatttgatcaagcaaatcttactgtaaaacttccagatggttatgaatttaaatttcctaatcgacttaatttggaagctattaattatatggctgctgatggagattttaaaattaaatgtgttgcttttgattaaggtaccaagctt

SEQ ID NO: 6 - DNA, 528 bp, Homo sapiens
  source 1..528: mol_type = unassigned DNA
atctctctcgggtggagtcttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaa

SEQ ID NO: 7 - DNA, 20 bp, synthetic construct
  misc_feature 1..20: note = Gal1 forward primer
  source 1..20: mol_type = other DNA
tcagcctggtcaaaggtgat

SEQ ID NO: 8 - DNA, 20 bp, synthetic construct
  misc_feature 1..20: note = Gal1 reverse primer
  source 1..20: mol_type = other DNA
tgaacctgggaaaagacagc

SEQ ID NO: 9 - DNA, 21 bp, synthetic construct
  misc_feature 1..21: note = IFN forward primer
  source 1..21: mol_type = other DNA
ggctgttactgccacggcaca

SEQ ID NO: 10 - DNA, 25 bp, synthetic construct
  misc_feature 1..25: note = IFN reverse primer
  source 1..25: mol_type = other DNA
caccatccttttgccagttcctcca

SEQ ID NO: 11 - DNA, 20 bp, synthetic construct
  misc_feature 1..20: note = IL5 forward primer
  source 1..20: mol_type = other DNA
acacagctgtccgctcaccg

SEQ ID NO: 12 - DNA, 21 bp, synthetic construct
  misc_feature 1..21: note = IL5 reverse primer
  source 1..21: mol_type = other DNA
tccacagtacccccacggaca

SEQ ID NO: 13 - DNA, 20 bp, synthetic construct
  misc_feature 1..20: note = IL10 forward primer
  source 1..20: mol_type = other DNA
ttccctgggtgagaagctga

SEQ ID NO: 14 - DNA, 20 bp, synthetic construct
  misc_feature 1..20: note = IL10 reverse primer
  source 1..20: mol_type = other DNA
cttcacctgctccactgcct

SEQ ID NO: 15 - DNA, 22 bp, synthetic construct
  misc_feature 1..22: note = IL17 forward primer
  source 1..22: mol_type = other DNA
ctccagaaggccctcagactac

SEQ ID NO: 16 - DNA, 24 bp, synthetic construct
  misc_feature 1..24: note = IL17 reverse primer
  source 1..24: mol_type = other DNA
agctttccctccgcattgacacag

SEQ ID NO: 17 - DNA, 19 bp, synthetic construct
  misc_feature 1..19: note = GADPH forward primer
  source 1..19: mol_type = other DNA
cagaacatcatccctgcat

SEQ ID NO: 18 - DNA, 21 bp, synthetic construct
  misc_feature 1..21: note = GADPH reverse primer
  source 1..21: mol_type = other DNA
gttcagctctgggatgacctt

SEQ ID NO: 19 - DNA, 239 bp, synthetic construct
  misc_feature 1..239: note = p170 promoter sequence
  source 1..239: mol_type = other DNA
aagcttagatctggaagtaattctagagctttgcatgtctataaaaaaatgggattccaaattaccgatatcaatatgcgaaaagaactatgaatatccactccatttttggttgccatttgttaacgctgcctcctctccctagtgctataataaaaatggcccattttggaacagacttctactattttgttgtctagtagcgggtctaaattagggtactgcagaaggaggtattc

SEQ ID NO: 20 - DNA, 84 bp, synthetic construct
  misc_feature 1..84: note = Usp45 secretion signal peptide sequence
  source 1..84: mol_type = other DNA
atgaaaaaaaagattatctcagctattttaatgtctacagtgatactttcagcagcagccccgttgtcaggtgtttacgctgcc

SEQ ID NO: 21 - DNA, 18 bp, synthetic construct
  misc_feature 1..18: note = spacer sequence
  source 1..18: mol_type = other DNA
cgggtctaaattagggta

(19) United States
(12) Patent Application Publication
(10) Pub. No.: US 2025/0041362 A1
(43) Pub. Date: Feb. 6, 2025

(54) GALECTIN-1 DELIVERY FOR THERAPEUTIC CONTROL OF INTESTINAL INFLAMMATION

(71) Applicants: UNIVERSIDAD NACIONAL DE ROSARIO, Rosario, Santa Fe (AR); FUNDACIÓN SALES, Ciudad Autónoma de Buenos Aires (AR); CONSEJO NACIONAL DE INVESTIGACIONES CIENTÍFICAS Y TÉCNICAS (CONICET), Ciudad Autónoma de Buenos Aires (AR)

(72) Inventors: Karina Valeria MARIÑO, Ciudad Autónoma de Buenos Aires (AR); Gabriel Adrián RABINOVICH, Ciudad Autónoma de Buenos Aires (AR); Victor Sebastian BLANCATO, Ciudad Autónoma de Buenos Aires (AR); Marta Alicia TOSCANO, Ciudad Autónoma de Buenos Aires (AR); Christian MAGNI, Ciudad Autónoma de Buenos Aires (AR); Luciano Gastón MOROSI, Ciudad Autónoma de Buenos Aires (AR)

(73) Assignees: UNIVERSIDAD NACIONAL DE ROSARIO, Rosario, Santa Fe (AR); FUNDACIÓN SALES, Ciudad Autónoma de Buenos Aires (AR); CONSEJO NACIONAL DE INVESTIGACIONES CIENTÍFICAS Y TÉCNICAS (CONICET), Ciudad Autónoma de Buenos Aires (AR)

(21) Appl. No.: 18/706,698

(22) PCT Filed: Nov. 1, 2022
(86) PCT No.: PCT/IB2022/060524

§ 371 Date: May 1, 2024

Related U.S. Application Data
(60) Provisional application No. 63/274,287, filed on Nov. 1, 2021.

(51) Int. Cl. A61K 35/747; A61K 38/00; A61K 45/06; A61P 1/00; A61P 29/00; A61P 37/06; C07K 14/47

(52) U.S. Cl. CPC A61K 35/747; A61K 45/06; A61P 1/00; A61P 29/00; A61P 37/06; C07K 14/4726; A61K 38/00

(57) ABSTRACT

The present invention relates to the delivery of galectins useful in the control of inflammation in diseases associated with inflammatory processes, such as intestinal inflammatory diseases. More particularly, the present invention relates to the delivery of Galectin-1 (Gal1) and Gal1-variants for controlling inflammation in such diseases. More specifically, the present invention relates to the treatment of Intestinal Inflammatory Diseases, in particular ulcerative colitis (UC) and Crohn's disease (CD). Even more specifically, the present invention relates to optimized lactic-acid bacterial strains, capable of producing and secreting considerable levels of human Gal1 and Gal1 variants for delivering galectin-1 into the inflamed tissue. Also, the present invention relates to expression cassettes comprising those Gal1 and variants thereof and the vectors wherein said expression cassettes are contained.

### FIELD OF THE INVENTION

The present invention relates to the delivery of galectins useful in the control of inflammation in diseases associated with inflammatory processes, such as intestinal inflammatory diseases. More particularly, the present invention relates to the delivery of Galectin-1 (Gal1) and Gal1-variants for controlling inflammation in such diseases. More specifically, the present invention relates to the treatment of Inflammatory Bowel Diseases, in particular ulcerative colitis (UC) and Crohn's disease (CD). Even more specifically, the present invention relates to optimized lactic-acid bacterial strains, capable of producing and secreting considerable levels of human Gal1 and variants thereof for delivering Galectin-1 into the inflamed tissue. Also, the present invention relates to expression cassettes comprising those Gal1 and variants thereof and to vectors wherein said expression cassettes are contained.

### BACKGROUND OF THE INVENTION

Chronic inflammatory diseases are considered a significant public health issue. These multifactorial pathological conditions involve a dysregulation of the immune system and account for more than 50% of deaths worldwide (Furman et al., 2019).

Also, different inflammation-mediated diseases affect a significant amount of the population in the industrialized world. Among them, intestinal diseases such as ulcerative colitis and Crohn's disease are the main forms of inflammatory bowel diseases (IBD). Also, other forms of inflammatory diseases have an immunologic origin.

Particularly, various autoimmune conditions and other idiopathic chronic inflammatory conditions (ICIC) represent a worldwide health problem. Understanding their pathogenesis is fundamental to improve prevention and develop useful therapies. Nonetheless, there is still much to know about the immune system function in order to address those issues.

An immune-mediated inflammatory disease (IMID) is a condition or disease without a definitive etiology but characterized by common pathways that lead to inflammation. Examples of autoimmune and/or inflammatory diseases include ankylosing spondylitis, psoriasis, psoriatic arthritis, Behcet's disease, rheumatoid arthritis, multiple sclerosis, encephalomyelitis, inflammatory bowel diseases (IBD), diabetes, celiac disease and allergy, as well as many cardiovascular, neuromuscular, and infectious diseases.

Autoimmune diseases could be caused either by genetic factors, hormone influence as by lifestyle and environmental factors, including persistent infections causing breakdown of immune tolerance. Also, depending on each disease, the symptoms that patients experience may be myocarditis, skin rash, impaired vision, pulmonary fibrosis, joint pain, among others. These inflammatory diseases target various organs and tissues making their treatment even harder. Current immunosuppressive therapies improve life quality, but they are not a definitive solution, and they also carry significant side effects. Furthermore, there are several non-responsive patients given the natural heterogeneity of these diseases.

Inflammatory bowel diseases (IBD) represent an important health problem, especially in industrialized countries. It is estimated that around 1.5 million people in the United States of America, about 2.2 million in Europe, and between 200,000 and 300,000 individuals in the rest of the world, are affected by inflammatory bowel diseases (Ananthakrishnan, 2015; Molodecky et al., 2012). According to estimated data by Fundación MAS VIDA (www.masvida.org.ar), 20,000-30,000 people suffer from these diseases in Argentina. However, considering the high incidence and frequency observed in specialized hospitals, this number is likely underestimated.

The main forms of IBD are ulcerative colitis (UC) and Crohn's disease (CD), which are considered typical diseases of the occidental way of living, predominantly in industrialized countries, Nordic latitudes and urban regions. Another form of IBD is called “undetermined” colitis, a term used when the symptoms observed do not strictly fall within any of the two above-mentioned forms. Annual incidence rates for UC are between 0-19.2 per 100,000 people in North America, and between 0.6-24.3 per 100,000 in Europe (Molodecky et al., 2012). In turn, incidence rates for CD are similar to those of UC, with up to 20.2 annual cases every 100,000 inhabitants in the US, and 0.3-12.7 new cases every 100,000 inhabitants in Europe. Global prevalence of IBD is estimated at 0.4%; nevertheless, both incidence and prevalence thereof have been increasing over the past decades (Ananthakrishnan, 2015; Molodecky et al., 2012).

Onset of IBD may occur at any age. In most of the cases, the diseases are diagnosed between the second and third decade of life. Approximately, a third of the cases are declared before the 20 years of age, mostly during adolescence, while only 4% start under the age of 5. Even though differences in etiology for pediatric-onset IBD and adult-onset IBD are still under discussion, the incidence of pediatric IBD (especially CD) has dramatically increased and emerged as a global challenge (Sýkora et al., 2018).

Both UC and CD are chronic diseases and involve a strong immunological component, characterized by outbreaks of inflammatory activity followed by remission (either spontaneous or treatment-induced) (Cosnes et al., 2011). IBD diagnosis is made, in most of the cases, during the first inflammatory peak. Even when symptoms are not present during remission stage, damage is still possible and increases with every outbreak. In extreme cases with tissue compromise, surgery is required for totally or partially removing the colon (colectomy) (Baumgart and Sandborn, 2012; Ordás et al., 2012).

While UC exclusively affects the colon mucosa, generally to a distal extent and in a continuous manner, CD is a chronic trans-mural inflammatory disorder (which may compromise smooth muscle tissue and serous membrane), affecting any segment of the gastrointestinal (GI) tract in a discontinuous fashion. When cases cannot be differentiated into either UC or CD, the term “undetermined colitis” is used, which encompasses various pathologies, considering these diseases are multifactorial, i.e., genetic predisposition, environmental factors, intestinal microbiota and an aberrant immune response are the elements which, combined, lead to the development thereof (Baumgart and Sandborn, 2012; Ordás et al., 2012). Nowadays, the general belief is that IBD arise as a consequence of a dysregulated inflammatory response against normal components of the microbiota, that—due to their proximity and mutualistic interactions-constitute auto-antigens per se (Kamada et al., 2013). However, several discussions remain in the state of the art regarding the uniformity of this triggering factor.

Based on certain histopathologic features, IBDs are classified into five different categories (Geboes, 2003): a) definite UC, acute inflammation with a severe distortion of crypts and diffuse depletion of calceiform cells, inflammation is continuous and limited to the mucosa. Vascularization is increased; b) probable UC. Diffuse mucosa inflammation, with only mild to moderate distortion of crypts and calceiform depletion; c) probable CD. Sub-mucosal or trans-mural focal inflammation with lymphocytic aggregates. Mucus retention with minimal acute inflammation; d) definite CD. The previous findings plus non-caseous granulomas or cracks (in surgery piece); and e) Undetermined Colitis. Simultaneous findings corresponding to both UC and CD.

Unfortunately, inflammatory bowel diseases do not have a cure yet. Therefore, the main objectives of current treatment are directed to inducing or maintaining a state of clinical remission, preserving suitable nutritional and developmental states and preventing or reducing the frequency and duration of relapses. Treatment should be personalized, adapted to severity of the disease, intestinal segment being affected, evolution history of the patient, nutritional state and existence of other medical complications.

Among the current therapies for IBD, the following are used: aminosalicylates (such as 5-aminosalicylic acid derivatives, 5-ASA), corticosteroids (the first to be used in the treatment of IBD, mainly prednisone and methylprednisolone, administered through either the oral or i.v. routes, and budesonide for local action), wide spectrum antibiotics, immunomodulators (i.e. azathioprine, 6-mercaptopurine, cyclosporine), and therapy with biologics (such as tumor necrosis factor (TNF)-neutralizing monoclonal antibodies, like infliximab, adalimumab, certolizumab pegol and golimumab). In that sense, the high percentage of primary or secondary resistance to anti-TNF highlighted the need for alternative therapeutic approaches and prompted the quest for new treatments targeting immunological regulators of inflammation. While neutralizing IFN-γ, IL-17 and IL-13 have shown promising results in colitis animal models, the clinical trials with fontolizumab (anti-IFN-γ) and secukinumab (anti-IL-17A), in CD, and tralokinumab (anti-IL-13) in UC did not show improvements in the clinical symptoms of these diseases (Danese et al., 2014; Hohenberger et al., 2017; Hueber et al., 2012; Reinisch, 2010). However, blockade of both IL-12 and IL-23 by Ustekinumab was approved for Crohn's disease and has shown promising results in UC (Fiorino et al., 2020). Clinical trials suggest the existence of a complex network of interactions between cytokines that regulate the inflammation in mucosa. In this context, specialists agree that multiple targets including inflammatory cytokines and effector T cells could have a greater therapeutic potential in IBD (Neurath, 2014).

Lastly, in recent years antibodies that block adhesion molecules have been developed to prevent lymphocyte recruitment, and thus decrease the severity of inflammation. In this context, monoclonal antibodies anti-α4β7 (vedolizumab), anti-integrin α4 (natalizumab) In turn, anti-integrin β7 (etrolizumab), and anti-MAdCAM-1 (SHP647, Duijvestein et al., 2019) are being tested in clinical trials with encouraging results (Hazel and O'Connor, 2020).

Although there is a variety of treatments available for IBD, they have shown severe side effects. Aminosalicylates for instance may lead to headache, nausea and diarrhea, but also to hemolysis and hepatic toxicity, among others. In turn, use of corticosteroids should be evaluated on a case-by-case basis, given that they may lead to growth alterations, liquid retention, and weight gain, redistribution of adipose tissue, hypertension, bone metabolism alteration, dermatological alterations, hyperglycemia, generalized immunosuppression, sub-capsular cataracts, myopathies and pseudo arthritis. Broad-spectrum antibiotics in CD, such as metronidazole, can produce headaches, hives, urethral or vaginal burning sensation and dyspepsia in up to 90% of the patients; such effects disappear when use is discontinued. However, the most significant adverse effect is peripheral neuropathy of the limbs, in up to 50% of the cases, which could be irreversible. Azathioprine (AZA) and 6-mercaptopurine (6-MP) may produce adverse effects of hypersensitivity mediated by the immune system (pancreatitis, fever, exanthema, arthralgias, general discomfort, nausea, diarrhea and hepatitis) and other associated effects such as leukopenia, thrombocytopenia and infections, as a consequence of the intra-erythrocyte accumulation of metabolites. Cyclosporine may produce hypertrichosis, neurotoxicity, nephrotoxicity, gingival hyperplasia, hypertension and risk of severe opportunistic infections. It can also be hepatotoxic and up to 30% of the patients may experience cholestasis. Finally, anti-TNF therapy, in addition to resistance mechanisms found in approximately 30% of patients (Hazel and O'Connor, 2020), could also lead to severe adverse reactions. These include systemic anaphylactic reactions and infectious complications in 5-30% of the patients. Recurrences of infectious diseases have been described during the treatment with biological therapies, especially tuberculosis and hepatitis B, therefore it is essential to rule out these pathologies before initiating treatment (Rosenblum et al., 2012; Hazel and O'Connor, 2020).

In view of the above-mentioned adverse effects, it is imperative to explore new therapeutic targets in IBD, which may promote, in combination with current therapies, resolution of local inflammatory response and induce normalization of intestinal architecture and functionality.

Similarly, there is also a need for alternative treatments for IMID that are effective in controlling the inflammation associated therewith, either alone or in combination therapy with known treatments.

The immune system involves several mechanisms dedicated to discriminate ‘self and non-self’ compounds in order to eliminate foreign antigens and/or develop tolerance. By this way, homeostasis is reached, evading autoimmune and chronic inflammatory diseases. This is achieved by maintaining certain balance between proinflammatory (Th1/Th17) and anti-inflammatory (Th2/Treg) cells. This immune homeostasis is shaped by several regulatory networks that involve cell surface glycosylation and lectin-glycan signaling pathways.

Galectins comprise a family of animal lectins capable of interacting with specific glycan structures present in diverse glycoconjugates on the cell surfaces and the extracellular matrix. Galectin-1 (Gal1), a member of this family with affinity for N-acetyl-lactosamine (LacNAc) residues present in complex N-glycans and core 2 O-glycans, has demonstrated anti-inflammatory activity and therapeutic potential in diverse models of autoimmune diseases (Sundblad et al., 2017; Toscano et al., 2018).

Although the immunomodulating properties of galectins have been demonstrated in several models of autoimmune diseases and inflammatory disorders, to date, only a few studies have been carried out to assess the role of these lectins in mucosal immunity and homeostasis, and in particular, in the intestinal mucosa.

It has been demonstrated that Gal1, -2, -3, -4 and -9 are expressed in the intestine under physiological conditions and localize in different areas. While Gal1 is mainly present in the Lamina propria, Gal2, Gal3, Gal4 and Gal9 are constitutively expressed by mouse intestinal epithelial cells. Gal3 and Gal4 are highly expressed in the epithelium of both small intestine and colon, while Gal2 was only identified at the colon level (Nio-Kobayashi et al., 2009). Regarding galectin functionality in intestinal physiopathology and the implications in local inflammatory processes, it was reported that administration of recombinant human Gal1 (rGal1) leads to reduced clinical, histopathological, and immunological manifestation in acute TNBS (2,4,6-trinitrobenzenesulfonic acid)-induced colitis in BALB/c mice, with decreased levels of pro-inflammatory cytokines (TNF, IL-1β, IL-12 and IFN-γ) both in plasma and colonic tissue. The anti-inflammatory properties of Gal lead to normalization of the intestinal mucosa architecture (Santucci et al., 2003). Recently, the present inventors have shown that rGal1 treatment also mitigated experimental colitis in a double instillation model of TNBS-induced colitis in C57Bl/6 mice, and animals lacking Gal1 exhibited exacerbated colitis and augmented mucosal CD8⁺ T cell activation in response to TNBS; this aggressive phenotype that was partially ameliorated by treatment with recombinant Gal1 (Morosi et al., 2021).

In patients with IBD, expression of this lectin is increased in inflamed areas of the colon (Papa Gobbi et al., 2016; Morosi et al, 2021). It should be noted that deficiency of Gal1 in mice leads to enlarged intestinal villi, as compared to wild-type mice, being this protein capable of modulating the enterocytes viability throughout their maturation from the crypts to the tip of the villi (Muglia et al., 2011). Also, inflammatory stimuli promote Gal1 binding to epithelial cells, both in biopsies of IBD patients and in murine intestinal inflammation models. Gal1 binding to enterocytes, besides influencing its survival, promotes the production of growth factors (EGF and TSLP) and the synthesis of anti-inflammatory cytokines (IL-10, IL-25 and TGF-β1) which in turn protect the intestinal epithelium from the inflammatory insult (Muglia et al., 2016).

Also, in experimental models of autoimmune disease, like collagen-induced arthritis, myelin-oligodendrocyte glycoprotein35-55-induced encephalomyelitis, diabetes, uveitis, and orchitis, Gal1 plays a central role, performing several immunoregulatory activities leading to the offset or amelioration of chronic inflammation. These effects are related to inhibition of proinflammatory cytokines, induction of tolerogenic DCs, expansion of Foxp3+ and Foxp3-Tregs and generation of alternatively activated “M2-type” macrophages by Gal1 (Toscano et al., 2018).

Particularly, in experimental autoimmune encephalomyelitis (EAE) it was found that astrocytes, present in the blood-brain barrier, expressed high amounts of Gal1 during both acute and chronic phases of the disease. Through the modulation of p38-MAPK, CREB, and NF-κB signaling pathways, Gal1 promoted deactivation of M1 microglia and upregulated a phenotype of M2 microglia activation, which promotes neuroprotection inhibiting axonal damage and its consequent neurodegeneration (Starossom et al. 2012).

Gal1 promotes secretion of growth factors and anti-inflammatory cytokines in epithelial cells, induces apoptosis of activated T_{H}1 and T_{H}17 lymphocytes and inhibits secretion of pro-inflammatory cytokines by dendritic cells (DCs) and T lymphocytes (Sundblad et al., 2017).

Additionally, expression of Gal1, Gal3, Gal4 and Gal9 was recently studied in a cohort of IBD patients and control individuals. By using a linear discriminant analysis (LDA), a molecular signature of galectins arose, which can be used to distinguish between normal mucosa and IBD inflamed mucosa, thus showing the degree of disease activity. This molecular signature of galectins could also be used for distinguishing samples from IBD patients and samples from other inflammatory bowel pathologies, such as colon graft rejection and/or active celiac disease (Papa Gobbi et al., 2016), thus evidencing the diagnostic and prognostic value of these molecules in inflammatory bowel pathologies. These findings suggest a central role for Gal1, as well as other galectins, as well as their specific ligands, as key mediators in the immunological homeostasis in the intestinal mucosa.

From a therapeutic point of view and considering Gal1 immunomodulatory functions, administration of this lectin suffers from several hurdles which prevent its immediate translation to therapy. These include the requirement of very high Gal1 concentrations to: a) overcome its sensitivity to oxidative inactivation and acidosis, b) its systemic distribution after administration and c) bias the equilibrium toward the immunosuppressive dimeric form. In experimental models, rGal1 is periodically injected in order to maintain an effective concentration in inflamed zones, and similarly to what has been described for other immunosuppressive agents in different autoimmune diseases, there could be side effects when considering its therapeutic potential (Boyapati et al., 2016). As a consequence, Gal1 highly stable variants with enhanced activity (International Patent Publication WO2016172319A1) have been generated previously, but a delivery of the Gal1 and variants thereof to inflamed tissue by oral administration with localized release of the therapeutic agents was still pending.

Therefore, there remains a need for alternative therapeutic compositions and methods for treatment of inflammatory diseases, both for providing an improvement to these disease conditions as well as a solution to the side-effects of the therapies known to-date.

In particular, with the aim of addressing the current drawbacks in available IBD treatments, and providing an effective therapeutic solution thereto, the present inventors have developed an alternative system for delivering Galectin-1 and variants thereof. The system proposed in the present invention is a lactic bacterial system consisting of a lactic acid bacteria (LAB) strain which expresses Galectin-1oroptimized variants, which are secreted to the extracellular medium in order to reduce inflammation.

Particularly, bacterial, non-classical protein expression systems such as Gram+ Lactococcus, a genus of LABs, are commonly used in the dairy industry (Song et al., 2017). Lactococcus bacteria are considered GRAS (generally regarded as safe), as they do not produce virulence factors and are not pathogenic for humans.

Additionally, many Lactococcus species are probiotics. Lastly, heterologous protein expression in LABs do not generate inclusion bodies in the bacterial protoplasm (Bermúdez-Humarán et al., 2013; Song et al., 2017).

Several groups have described LABs genetically modified to produce anti-inflammatory proteins such as IL-10 and IL-27 and have demonstrated therapeutic effects in experimental models of inflammatory intestinal diseases (Hanson et al., 2014; Steidler et al., 2000).

The European application EP3464332A1 discloses a polypeptide sequence for treating intestinal inflammatory disease that involves the use of a Bifidobacterium breve bacterial strain.

The present invention provides a therapeutic solution to inflammatory diseases, particularly, intestinal inflammatory diseases.

### SUMMARY OF THE INVENTION

In view of the above, an exhaustive experimental research focusing on the development of new therapeutic approaches for intestinal inflammatory diseases has been performed.

The present invention provides a bacterial delivery system for expression and secretion of human Galectin-1 (Gal1) and variants thereof having their nucleotide sequences optimized for its expression in Lactococcus lactis, as depicted in SEQ ID NO: 1-5. These sequences comprise the optimized sequences for human Galectin-1 or variants thereof (under the control of a promoter sequence) and the sequence of a signal secretion peptide at its 5′-end, wherein the signal secretion peptide is usp45, as shown in FIG. 1. The sequence of the signal secretion peptide usp45 is depicted herein as SEQ ID NO: 20. The sequence of endogenous Galectin-1 (WT human Gal1; i.e., Homo sapiens galectin-1 (LGALS1), NCBI Reference Sequence: NM_002305.4, the sequence of which is depicted herein as SEQ ID NO: 6) was optimized for its expression in the mentioned bacterial system.

Therefore, an object of the present invention is to provide a nucleotide construct comprising a promoter sequence and an insert, wherein:
said insert consists of a galectin-1 nucleotide sequence fused to a signal secretion peptide, wherein the galectin-1 nucleotide sequence is selected from the group consisting of SEQ ID NO: 1-5, and wherein said secretion peptide is usp45 of SEQ ID NO: 20; and
said promoter sequence has a sequence as depicted in SEQ ID NO: 19 (see FIGS. 2-6).

Another object of the present invention is to provide a vector comprising the construct of the invention, wherein the vector is the pNZ8048 plasmid.

According to another object of the present invention, a transformed host cell capable of secreting sequence-optimized human Gal1 or variants thereof is also provided. In particular, said host cell is a lactic acid bacterium.

According to embodiments of this object of the present invention, said transformed host cell is Lactococcus lactis. Other possible host cells useful for the present invention are Bifidobacterium breve, Lactobacillus paracasei LS2, and Faecalibacterium prausnitzii.

According to a preferred embodiment of the invention, the transformed host cell is Lactococcus lactis (NZ9000).

In a further preferred embodiment of the present invention, the Lactococcus lactis strains used for the invention are those deposited on Nov. 11, 2020 at the Chilean Collection of Microbial Genetic Resources (CChRGM), under the Budapest Treaty with the following Accession Numbers RGM3045-3049.

The present invention also envisages a pharmaceutical composition comprising the transformed Lactococcus lactis strain designed according to the present invention, and pharmaceutically acceptable excipients. According to a preferred embodiment of the present invention, the composition is suitable for oral administration.

According to another object of the present invention, a method for delivering said Gal1 and its variants is provided, wherein the method comprises orally administering the Lactococcus lactis strain generated according to the invention, so that the Gal1 or variants thereof are expressed and secreted to extracellular medium for inflammation control.

It is also envisaged in the present invention a method for controlling inflammation in a subject suffering from IBD, comprising orally administering a composition including a transformed Lactococcus lactis strain described herein, which expresses the optimized Galectin-1 or variants according to the invention.

According to a preferred object of the invention, the IBD is selected from ulcerative colitis and Crohn's disease.

It is also another object of the present invention the use of a transformed Lactococcus lactis strain expressing Galectin-1 (Gal1) or variants thereof according to the invention for manufacturing a medicament for the treatment of Inflammatory Bowel Diseases (IBD).

According to a particular object, the present invention provides a food product or food supplement comprising or consisting of a transformed host cell according to the invention deposited on Nov. 11, 2020 at the Chilean Collection of Microbial Genetic Resources (CChRGM), under the Budapest Treaty with the following Accession Numbers RGM3045-3049, wherein the food product/supplement is in a form suitable for oral administration. According to a preferred embodiment, the food product/supplement may be in the form of a lactose-free yoghurt, or a bacterial suspension, or a pill containing dried-bacteria. Preferably, the food product/supplement is in the form of a bacterial suspension.

### BRIEF DESCRIPTION OF THE DRAWINGS

The following figures form part of the present specification and are included to further illustrate certain aspects of the present invention, without limiting the scope thereof.

FIG. 1. Schematic representation of the constructions used in the present invention wherein the promoter P170 is located upstream the secretion signal peptide (usp45), that is upstream the endogenous Galectin-1 or variants thereof with their sequences optimized for its expression in Lactococcus lactis.

FIG. 2. Circular map of the construction containing endogenous Galectin-1 of the present invention, wherein the promoter p170 is located at the 5′-end of the secretion signal peptide (usp45) which is located at the 5′-end of endogenous Galectin-1, with its sequence optimized for expression in Lactococcus lactis. Also shown are restriction enzymes cutting site (BgIII, XbaI, EcoRV, PstII, NdeI, NcoI, KpnI and HindIII), repC and repA replication proteins and chloramphenicol resistant cassette (cm).

FIG. 3. Circular map of the construction containing Gal1 variant SG1 of the present invention, wherein the promoter p170 is located at the 5′-end of the secretion signal peptide (usp45) which is located at the 5′-end of variant SG1, with its sequence optimized for expression in Lactococcus lactis. Also shown are restriction enzymes cutting site (BgIII, XbaI, EcoRV, PstII, NdeI, NcoI, KpnI and HindIII), repC and repA replication proteins and chloramphenicol resistant cassette (cm).

FIG. 4. Circular map of the construction containing Gal1 variant SG2 of the present invention, wherein the promoter p170 is located at the 5′-end of the secretion signal peptide (usp45) which is located at the 5′-end of variant SG2, with its sequence optimized for expression in Lactococcus lactis. Also shown are restriction enzymes cutting site (BgIII, XbaI, EcoRV, PstII, NdeI, NcoI, KpnI and HindIII), repC and repA replication proteins and chloramphenicol resistant cassette (cm).

FIG. 5. Circular map of the construction containing Gal1 variant SG3 of the present invention, wherein the promoter p170 is located at the 5′-end of the secretion signal peptide (usp45) which is located at the 5′-end of variant SG3, with its sequence optimized for expression in Lactococcus lactis. Also shown are restriction enzymes cutting site (BgIII, XbaI, EcoRV, PstII, NdeI, NcoI, KpnI and HindIII), repC and repA replication proteins and chloramphenicol resistant cassette (cm).

FIG. 6. Circular map of the construction containing Gal1 variant SG4 of the present invention, wherein the promoter p170 is located at the 5′-end of the secretion signal peptide (usp45) which is located at the 5′-end of variant SG4, with its sequence optimized for expression in Lactococcus lactis. Also shown are restriction enzymes cutting site (BgIII, XbaI, EcoRV, PstII, NdeI, NcoI, KpnI and HindIII), repC and repA replication proteins and chloramphenicol resistant cassette (cm).

FIGS. 7A-7B. rGal1 synthesis and secretion by L. lactis pNZ, L. lactis pNZ-Gal1 and L. lactis pNZ-usp:Gal1 at different induction times with nisin, measured by ELISA. (7A) Intracellular fraction, post-lysis. (7B) Conditioned media. Results are representative of three independent experiments.

FIG. 8. rGal1 synthesis and secretion by L. lactis pNZ-usp:Gal1 at different induction times with nisin. Detection of rGal1 in intracellular (Intracell) and conditioned media (secreted) fractions by western-blot analysis using a polyclonal anti-Gal1 antibody. rGal1: recombinant human Gal1 as a positive control.

FIGS. 9A-9C. (9A) Protocol scheme for in vivo administration of L. lactis pNZ-usp:Gal1 in a TNBS-induced acute colitis model. Colitis was induced and L. lactis pNZ-usp:Gal1 (3.2.10⁸ CFU) were administered by oral gavage every two days. (9B) Weight curves and colon weight/length at sacrifice of wild-type (WT) mice treated with TNBS and treated with L. lactis pNZ-usp:Gal1.*p<0.05 ANOVA followed by Tukey post-test. Figure shows mean±SEM from a representative experiment (3 mice/group). (9C) Weight curves and colon weight/length at sacrifice of Gal1 deficient (Lgals1^{-/-}) mice treated with TNBS and treated with L. lactis pNZ-usp:Gal1.*p<0.05 ANOVA followed by Tukey post-test. Figure shows mean±SEM from a representative experiment (3 mice/group).

FIGS. 10A-10C. rGal1 expression in genetically modified L. lactis. Three L. lactis strains were originally tested: pNZ (L. lactis with empty vector, pNZ), L. lactis genetically modified to produce intracellular Gal1 (pNZ-Gal1) and genetically modified to produce secreted forms of Gal1 (pNZ-usp:Gal1). Three additional strains were generated and added to experimentation: pNZ-Gal1-Opt (codon optimized production of intracellular Gal1), pNZ-usp:Gal1-Opt (secreted Gal1, codon optimized, with nisin as inductor), and p170-usp:Gal1-Opt (secreted rGal1, codon-optimized, induction by low pH). (10A) Detection of rGal1 produced by said additional strains of L. lactis by western blot. Intracellular fraction and conditioned M17G medium (Secreted) are shown. (10B) Intracellular rGal1 measured by ELISA and normalized by total lysate protein. Data show median±SD of 2 independent experiments. (10C) Secreted rGal1 measured by ELISA and normalized by the optical density of cells in culture (OD_{600 nm}). Data show median±SD of 2 independent experiments.

FIG. 11. L. lactis p170-usp:Gal1-Opt produces rGal1 in higher levels than L. lactis pNZ-usp:Gal1. Quantification of rGal1 was done by ELISA in conditioned media from 6 hours cultures. Results are obtained from three independent experiments.

FIG. 12. rGal1 and its variants (SG1, SG2, SG3, SG4) expression in L. lactis, genetically modified to produce secreted forms of this lectin. Induction of expression was achieved as previously described (p170-usp:Gal1-Opt, secreted rGal1, codon-optimized, induction by low pH). Detection of rGal1 and variants produced by said strains of L. lactis by western blot. Total cellular extract and conditioned M17G medium (Secreted) are shown at different times of culture (hours).

FIGS. 13A-13B. Recombinant human galectin-1 (rGal1) produced and secreted by L. lactis p170-usp:Gal1-Opt binds to immobilized lactose, confirming ligand recognition and binding. (13A) Western blot analysis of lactosyl-sepharose fractions. rGal1, recombinant human Gal1 as positive control; CM, conditioned media (M17G) from 6 hours culture; CM-PBS, conditioned media (M17G) from a 6-hour culture diluted with PBS previous to column loading; W, washes with PBS after loading; F1-F4, fractions eluted with lactose 150 mM. (13B) Semi-quantitative densitometry for western-blot bands.

FIGS. 14A-14C. Variants of human Galectin-1 (SG1, SG2, SG4) produced and secreted by L. lactis p170-usp:Gal1-Opt bind to immobilized lactose, confirming ligand recognition and binding. (14A) Variant SG1, western blot analysis of lactosyl-Sepharose fractions. rGal1, recombinant human Gal1 as positive control; CM, conditioned media (M17G) from 6 hours culture; CM-PBS, conditioned media (M17G) from a 6-hour culture diluted with PBS previous to column loading; F2-F5, fractions eluted with lactose 150 mM. (14B) Variant SG2, western blot analysis of lactosyl-sepharose fractions. rGal1, recombinant human Gal1 as positive control; F1-F5, fractions eluted with lactose 150 mM. (14C) Variant SG4, western blot analysis of lactosyl-Sepharose fractions. F2-F6, fractions eluted with lactose 150 mM; rGal1, recombinant human Gal1 as positive control.

FIGS. 15A-15B. L. lactis p170-usp:Gal1-Opt supernatant together with TNFα favors wound closure in HT-29 colon adenocarcinoma cell line in an in vitro scratch assay. (15A) Representative photomicrographs of two experimental groups, HT-29 epithelial cells treated with TNFα (50 ng/ml) and the supernatant of either L. lactis p170-usp:Gal1-Opt (left) or L. lactis pNZ (right). Photographs were taken at 10× magnifications with an Olympus CX31 microscope. (15B) Wounded area measured in pixels in each condition, at least 4 fields were analyzed for each assay-triplicate. Conditions from left to right: rGal1 (1 μM), medium alone, supernatant of L. lactis p170-usp:Gal1-Opt, supernatant of L. lactis pNZ, rGal1 (1 μM) and TNFα (50 ng/ml), TNFα (50 ng/ml) alone, supernatant of L. lactis p170-usp:Gal1-Opt and TNFα (50 ng/ml), supernatant of L. lactis pNZ and TNFα (50 ng/ml). Area of the wound was measured using ImageJ software and the Wound healing size tool (Suarez-Arnedo et al, 2020). **p<0.01 one-way ANOVA and Šídák's multiple comparisons test.

FIG. 16. Administration of L. lactis p170-usp:Gal1-Opt alleviates colitis in Lgals1^{-/-} mice. (16A) Representative weight curves for mice treated with TNBS and daily treated with 1.10⁹ CFU of L. lactis p170-usp:Gal1-Opt or L. lactis pNZ (control strain). ***p<0.001 repeated measures ANOVA followed by Tukey post-test, comparing evolution of both curves. (16B) Colon weight/length at sacrifice. *p<0.05 unpaired Student t-test. Figure shows mean±SEM from a representative experiment (8 mice/group).

FIG. 17. L. lactis p170-usp:Gal1-Opt delivers rGal1 through the digestive tract of Lgals1^{-/-} mice. Quantification of rGal1 by ELISA normalized to total protein content in duodenum, jejunum, ileum and colon. Figure shows mean±SEM from a representative experiment (8 mice/group).

FIGS. 18A-18B. Administration of L. lactis p170-usp:SG1-Opt or control strain L. lactis pNZ in Lgals1^{-/-} mice coursing TNBS induced-colitis. (18A) Representative weight curves for mice treated with TNBS and daily treated with 1.10⁹ CFU of L. lactis p170-usp:SG1-Opt or L. lactis pNZ (control strain). Repeated measures ANOVA followed by Tukey post-test, comparing evolution of both curves, p-value non-significant. (18B) Colon weight/length at sacrifice. Unpaired Student t-test, p non-significant, Figure shows mean±SEM from a representative experiment (11 mice/group at day 0, 4-5 mice/group at day 10).

FIGS. 19A-19B. Administration of L. lactis p170-usp:Gal1-Opt or control strain in WT mice coursing TNBS-induced colitis. (19A) Representative body weight curves for mice treated with TNBS and daily treated with 1.10⁹ CFU of L. lactis p170-usp:Gal1-Opt or L. lactis pNZ (control strain). Repeated measures ANOVA followed by Tukey post-test, comparing evolution of both curves, p non-significant. (19B) Colon weight/length at sacrifice. Unpaired Student t-test, p-value non-significant. Figure shows mean±SEM from a representative experiment (9 mice/group at day 0, 4-6 mice/group at day 10).

FIGS. 20A-20B. Characterization of the cytotoxic T cell population in Lgals1^{-/-} mice coursing TNBS-induced colitis and treated (or not) with L. lactis p170-usp:SG1-Opt. Proportions of CD8⁺ T cells in Lamina propria (LP) (20A) and mesenteric lymph nodes (20B) in Lgals1^{-/-} mice coursing intestinal inflammation daily treated with 1.10⁹ CFU of L. lactis p170-usp:SG1-Opt or control strain (L. lactis pNZ). Unpaired Student t-test. Figures show mean±SEM from a representative experiment (11 mice/group at day 0, 4-5 mice/group at day 10).

FIGS. 21A-21B. Characterization of the myeloid cell compartment in Lgals1^{-/-} mice coursing colitis and receiving L. lactis p170-usp:SG1-Opt or control strain L. lactis pNZ. Proportions of macrophages Ly6^{low}MHCII^{hi} (21A) and neutrophils CD11b⁺Ly6G⁺ (21B) in Lamina propria (LP) in Lgals1^{-/-} mice coursing intestinal inflammation daily treated with 1.10⁹ CFU of L. lactis p170-usp:SG1-Opt or control strain (L. lactis pNZ). Unpaired Student t-test, p-value non-significant. Figures show mean±SEM from a representative experiment (11 mice/group at day 0, 4-5 mice/group at day 10).

FIGS. 22A-22B. Characterization of the cytotoxic T cell population in WT mice coursing TNBS-induced colitis treated (or not) with L. lactis p170-usp:Gal1-Opt. Proportions of CD8⁺ T cells in Lamina propria (LP) (22A) and mesenteric lymph nodes (22B) in WT mice coursing intestinal inflammation daily treated with 1.10⁹ CFU of L. lactis p170-usp:Gal1-Opt or control strain (L. lactis pNZ). Unpaired Student t-test, p-value non-significant. Figures show mean±SEM from a representative experiment (9 mice/group at day 0, 4-6 mice/group at day 10).

FIGS. 23A-23D. Administration of L. lactis p170-usp:Gal1-Opt in Lgals1^{-/-} mice alters colonic cytokine expression. Measurement of colonic IL-5 (23A), IL-10 (23B), IL-17 (23C) and IFN-γ (23D) levels by RT-qPCR in Lgals1^{-/-} mice coursing intestinal inflammation daily treated with 1.10⁹ CFU of L. lactis p170-usp:Gal1-Opt or control strain (L. lactis pNZ). Box representation of median (line), first and third quartile (box limits), minimum and maximum (bars) from three different experiments (8 mice/group). *p<0.05 unpaired Student t-test.

FIGS. 24A-24D. Colonic cytokine expression in Lgals1^{-/-} mice coursing TNBS-induced colitis and treated (or not) with L. lactis p170-usp:SG1-Opt. Measurement of colonic IL-5 (24A), IL-10 (24B), IL-17 (24C) and IFN-γ (24D) levels by RT-qPCR in Lgals1^{-/-} mice coursing intestinal inflammation daily treated with 1.10⁹ CFU of L. lactis p170-usp:SG1-Opt or control strain (L. lactis pNZ). Box representation of median (line), first and third quartile (box limits), minimum and maximum (bars) from three different experiments (11 mice/group at day 0, 4-5 mice/group at day 10). Unpaired Student t-test, p-value non-significant.

FIGS. 25A-25D. Administration of L. lactis p170-usp:Gal1-Opt in WT mice alters colonic cytokine expression. Measurement of colonic IL-5 (25A), IL-10 (25B), IL-17 (25C) and IFN-γ (25D) levels by RT-qPCR in WT mice coursing intestinal inflammation daily treated with 1.10⁹ CFU of L. lactis p170-usp:Gal1-Opt or control strain (L. lactis pNZ). Box representation of median (line), first and third quartile (box limits), minimum and maximum (bars) from three different experiments (9 mice/group at day 0, 4-6 mice/group at day 10). **p<0.01 unpaired Student t-test.

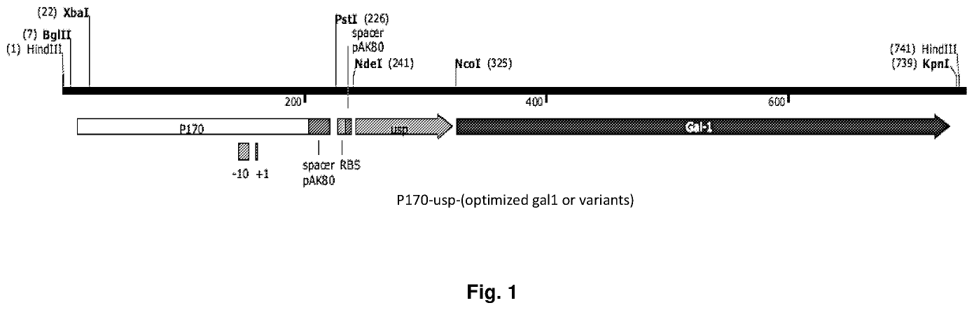

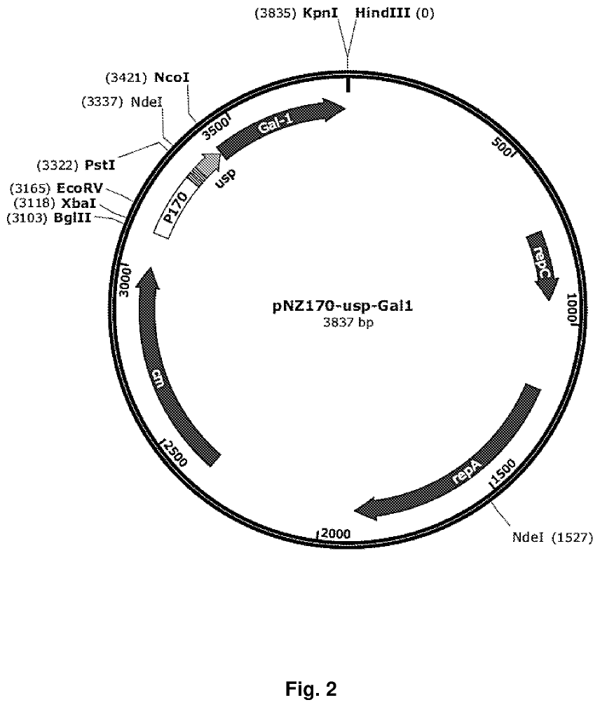

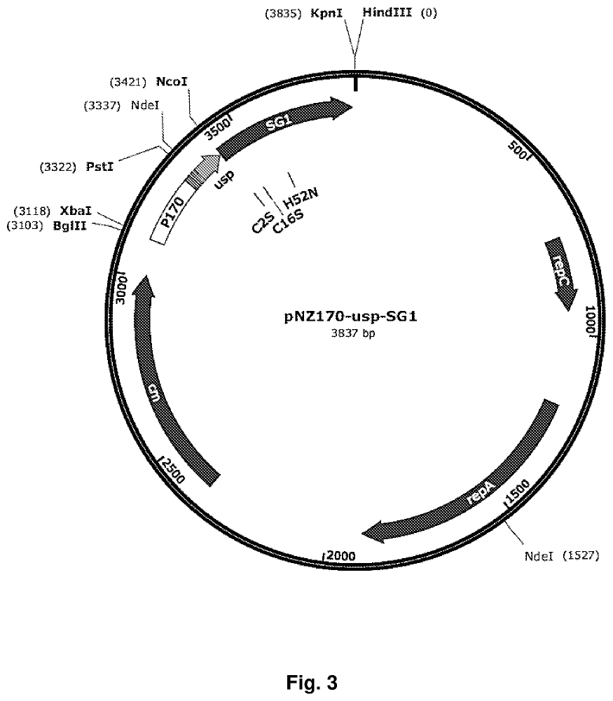

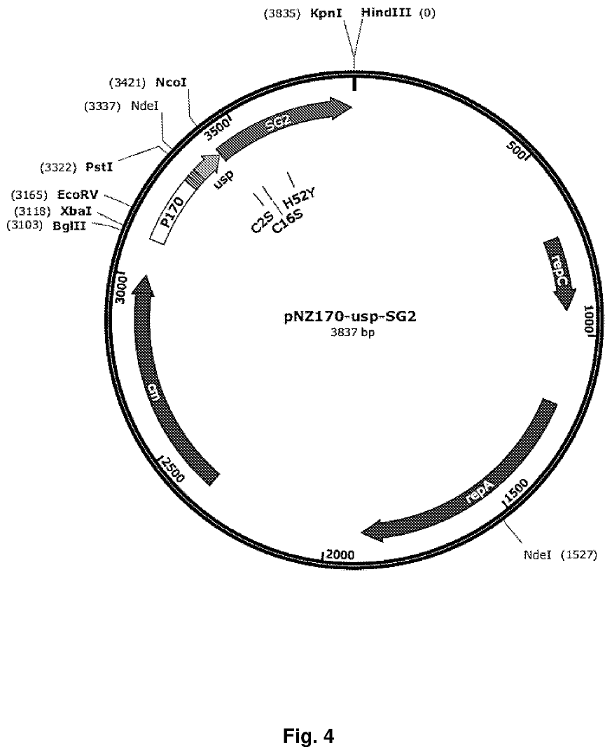

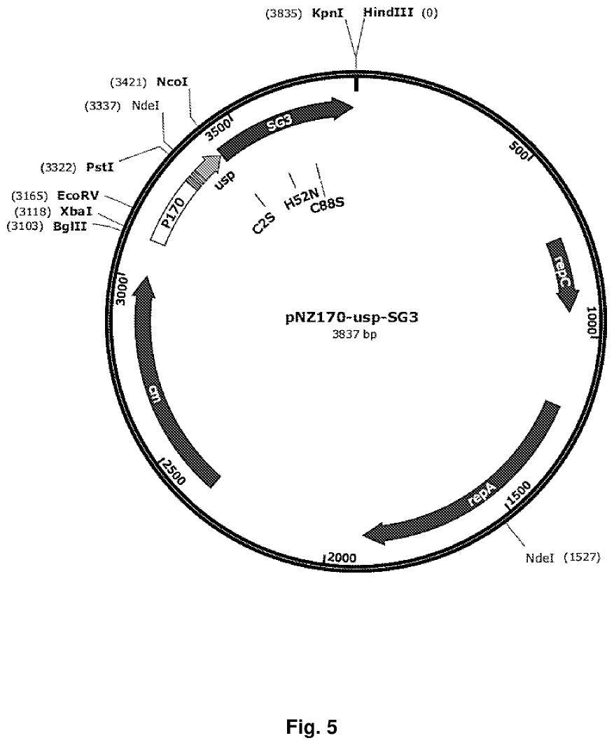

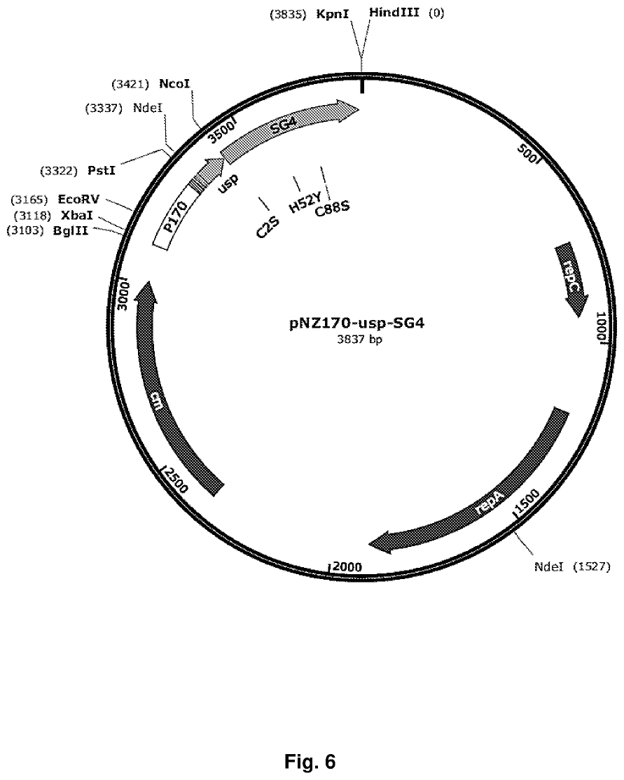

### DETAILED DESCRIPTION OF THE INVENTION

The present invention discloses bacterial delivery system/s for expression and secretion of sequence-optimized galectins useful in the control of inflammation, and inflammatory-mediated diseases.

More specifically, the bacterial delivery systems disclosed in the present invention are useful in the treatment of Inflammatory Bowel Diseases (IBD), in particular ulcerative colitis (UC) and Crohn's disease (CD). Throughout this specification, the terms “Inflammatory Bowel Disease/s” and “Intestinal Inflammatory Disease/s” are used interchangeably.

As discussed above herein, the present inventors sought for a targeted and selective delivery of sequence-optimized Gal1 (Gal1-Opt) or its variants to inflamed tissues, taking into consideration its immunoregulatory activity.

More specifically, lactic-acid bacterial (LAB) strains, capable of producing and secreting considerable levels of human Gal1 or variants thereof into the intestinal lumen (inflamed tissue) for an effective control of inflammation, have been developed.

These strains were obtained by transforming Lactococcus lactis with constructs comprising a promoter sequence and an insert, wherein
said insert consists of a galectin-1 nucleotide sequence fused to a signal secretion peptide, wherein the galectin-1 nucleotide sequence is selected from the group consisting of SEQ ID NO: 1-5, and wherein said secretion peptide is usp45 of SEQ ID NO: 20; and
said promoter sequence has a sequence as depicted in SEQ ID NO: 19.

The strains thus obtained are able to express either human Gal1 (hereinafter, Gal1) or the variants thereof noted as SG1, SG2, SG3 and SG4. The codifying sequences for each of these peptides have been optimized for their expression in L. lactis and are as follows: SEQ ID NO: 1 (Gal1), SEQ ID NO: 2 (SG1), SEQ ID NO: 3 (SG2), SEQ ID NO: 4 (SG3), and SEQ ID NO: 5 (SG4).

Said strains have been deposited on Nov. 11, 2020 at the Chilean Collection of Microbial Genetic Resources (CChRGM) under the Budapest Treaty and have Accession Number/s RGM3045-3049. The strain deposited under Accession Number RGM3045 expresses human Gal1. The strain deposited under Accession Number RGM3046 expresses SG1. The strain deposited under Accession Number RGM3047 expresses SG2. The strain deposited under Accession Number RGM3048 expresses SG3. The strain deposited under Accession Number RGM3049 expresses SG4.

The present invention also relates to the constructs which allow the expression of Gal1 as well as SG1, SG2, SG3 and SG4. Accordingly, another object of the present invention is to provide a nucleotide construct comprising a promoter sequence and an insert, wherein
said insert consists of a galectin-1 nucleotide sequence fused to a signal secretion peptide, wherein the galectin-1 nucleotide sequence is selected from the group consisting of SEQ ID NO: 1-5, and wherein said secretion peptide is usp45 of SEQ ID NO: 20; and
said promoter sequence has a sequence as depicted in SEQ ID NO: 19.

The present invention also relates to vectors comprising the constructs of the invention. Preferably, the vector comprising the construct of the invention is the pNZ8048 plasmid.

The constructs of the invention may be expressed in host cells suitable for such an expression. Particularly, the invention relates as well to transformed host cells which express the vector comprising the construct of the invention, preferably, wherein the vector is the pNZ8048 plasmid.

The host cells suitable for expressing the aforementioned vector are bacterial cells. A preferable host cell according to the present invention is a lactic acid bacterium, more preferably, selected from the group consisting of Lactococcus lactis, Bifidobacterium breve, Lactobacillus paracasei LS2, Faecalibacterium prausnitzii, L. plantarum, L. salivarius lactic-acid bacteria, as long as the system allows it, which a person of skill in the art will be able to appreciate. Most preferably, the transformed host cell according to the invention is a cell of Lactococcus lactis strain NZ9000.

The inventors have found that the oral administration of the bacterial strains of the invention allow for the effective control of inflammation in experimental models of inflammatory diseases, such as inflammatory bowel diseases (IBD).

The inventors have surprisingly found that the bacterial strains of the invention are especially able to provide a localized delivery of Gal1, SG1, SG2, SG3 or SG4 to sites in need of anti-inflammatory effect within the bowel. Therefore, the strains of the invention are particularly useful for intestinal inflammation.

Correspondingly, the present invention also relates to a method for controlling inflammation in a subject suffering from an inflammation-mediated disease comprising administering a therapeutically effective amount of any of the Lactococcus lactis strains of the invention to a subject in need thereof. Preferably, the inflammation-mediated disease is an IBD.

The IBD which may be treated by the present invention include, but are not limited to, ulcerative colitis and Crohn's disease.

When treating IBD, the strains of the present invention may be administered in combination with at least one additional therapeutic agent known for such a treatment. In a preferable embodiment, said at least one additional therapeutic agent comprises at least one immunosuppressive drug. The coadministration of the transformed bacterial strains of the invention in combination with the at least one additional therapeutic agent may be sequential or simultaneous.

The bacterial strains of the invention may also be used for treating immune-mediated inflammatory diseases (IMID). Therefore, yet another aspect of the present invention is to provide a method for controlling inflammation in a subject suffering from an IMID comprising administering a therapeutically effective amount of any of the Lactococcus lactis strains of the invention to a subject in need thereof. The IMID which may be treated by the present invention include, but are not limited to, rheumatoid arthritis and multiple sclerosis.

Another aspect of the invention is to provide a pharmaceutical composition comprising a transformed Lactococcus lactis strain according thereto. The pharmaceutical compositions of the invention comprise an amount of Lactococcus lactis which is effective to provide a therapeutic effect, as well as pharmaceutically acceptable excipients. The pharmaceutical composition of the invention may be prepared by methods and techniques which are well known for a person of skill in the art. In a preferred embodiment, the pharmaceutical composition is for oral administration.

Oral pharmaceutical compositions included within the scope of the present invention include such typical excipients as, for example, pharmaceutical grades of mannitol, starch, magnesium stearate, sodium saccharine, cellulose, magnesium carbonate and the like. The compositions take the form of solutions, suspensions, tablets, pills, capsules, sustained release formulations or powders, and any other form currently used, including cremes, lotions, mouthwashes, inhalants and the like.

It is also envisioned by the present invention to provide a food product or food supplement comprising or consisting of a lyophilized or dehydrated form of a transformed bacterial strain expressing Gal1, SG1, SG2, SG3 or SG4 according to the invention, for the prevention and treatment of inflammation-mediated diseases.

In a preferred embodiment, said inflammation-mediated diseases are IBD.

In a particularly preferred embodiment, the IBD is selected from ulcerative colitis and Crohn's disease.

The food products according to the invention may be selected from any food product used in the art for aiding in the prevention this kind of diseases. However, the bacterial strains of the present invention express proteins which have a particular specificity for lactose-type structures. Therefore, in a preferred embodiment, the food product of the invention is lactose-free.

Particularly, the food product is selected from the group consisting of acidified lactose-free milk, lactose-free yogurt, lactose-free frozen yogurt, lactose-free milk powder, lactose-free milk concentrate, lactose-free cheese spreads, dressings and beverages, bacterial suspension or a pill containing dried-bacteria. Preferably, the food product is in the form of a bacterial suspension.

In a particular embodiment, the food product of the invention may be used as a supplement in lactose-free fortified milk and milk-derived products, particularly those selected from milk powder, milk concentrate, yoghurt and cream cheese.

Also, oral pharmaceutical compositions and food products comprising mixtures of the strains mentioned herein are within the scope of the invention. The bacterial strains according to the present invention can be prepared in various dosage forms.

Below, some experimental assays are described for better understanding how to take into practice the present invention.

The following protocols, preparation of media, processing and characterization procedures were used in the examples provided for illustrating the present invention:

### In Vitro Assays

Culture Media for Lactococcus lactis

The M17 media was prepared (1% w/v bactopeptone; 0.25% w/v yeast extract; 0.5% w/v beef extract; 0.05% w/v ascorbic acid, 1.9% w/v β-glycerophosphate) and sterilized by autoclave. Upon its use, the media was supplemented with glucose (0.5% v/v), MgCl₂ (5 mM) and MnCl₂ (1.5 mM), from concentrated stock solutions, sterilized through 0.22 μm-pore size filters (Millipore). Finally, chloramphenicol (Sigma) was added to a final concentration of 5 μg/ml. This culture media is known as M17G.

All the Lactococcus lactis NZ9000 (GenBank database Accession number CP002094) strains used were grown in M17G media, without shaking at 30° C., in previously sterilized, closed vessels. Several aliquots of each of the strains were stored at −70° C. in M17G media with glycerol 20% v/v.

Preparation of Protein Extracts from Bacteria and Conditioned Media

Preparation of proteins from L. lactis was carried out from 1.5 ml culture aliquots. Samples were separated by centrifugation at 10000×g for 10 minutes, and cellular precipitates and supernatants (conditioned media) were treated separately.

Conditioned media were either used unmodified or the proteins were precipitated with trichloroacetic acid (TCA) at a final concentration of 20% v/v, incubated for 20 minutes in ice and centrifuged for 15 minutes at 15000×g and 4° C.

Cellular precipitates were resuspended in TSS-lysozyme buffer (20 mM Tris-HCl pH 8.0; 150 mM NaCl; 1 mM EDTA; 0.5 mg/ml lysozyme) supplemented with 1 mM PMSF and 10 mM 2-mercaptoethanol, and incubated for 30 minutes at 37° C. Subsequently, they were lysed by sonication.

Protein concentration of the cellular extracts was determined by using the Micro BCA kit (Pierce) following the manufacturer's instructions. Protein concentration was determined by measuring absorbance at a wavelength of 550 nm in a plate spectrophotometer (Labsystems Multiskan), carrying out the same procedure with a standard BSA curve (0.5-100 μg/ml).

Western Blot for Gal1 and Variants from Protein Extracts

30 μg total protein were prepared from the protein extracts obtained as described hereinabove, from the bacterial lysis or from total protein precipitated in conditioned media in sample buffer 2× (Laemmli Sample Buffer; Bio-Rad) in the presence of 355 mM 2-mercaptoethanol as a reducing agent, incubation at 100° C. for 5 minutes for denaturing the proteins present. 1.5 mm-thick 10%-15% polyacrylamide gels (SDS-PAGE) were prepared. After seedling the samples, the electrophoretic run was carried out in an electrophoresis cell (Bio-Rad) at a constant voltage of 150 V, in electrophoretic buffer (25 mM Tris pH 8.3; 192 mM glycine; 0.1% w/v SDS) for 1 hour approximately. Subsequently, protein was transferred from the gel to nitrocellulose membranes (Amersham Biosciences) at 200 mA constant for 60 minutes, using a Mini transblot equipment (Bio-Rad). Then, the nitrocellulose membranes were incubated in blocking buffer (0.15 M NaCl; 50 mM Tris; 0.1% v/v Tween-20 and 5% w/v skimmed milk) for 1 hour at room temperature in an orbital shaker. Blocked membranes were washed with TBS-T buffer (0.15 M NaCl; 50 mM Tris; 0.1% v/v Tween-20) and then incubated with primary antibody (purified rabbit IgG anti-mouseGal1) diluted in TBS-T 1% w/v skimmed milk, at 4° C. overnight in an orbital shaker. Then, the membranes were washed with TBS-T buffer and incubated for 1 hour with the peroxidase-conjugated secondary anti-rabbit antibody (1/3000) (Bio-Rad) in TBS-T 1% w/v milk for 1 hour. Detection was performed with the chemiluminescent kit ECL (Amersham Biosciences) using a G-Box equipment (Syngene).

### Determination of Gal1

The concentration of Gal1 present in the bacterial extracts and unmodified conditioned media obtained hereinabove, was determined through an ELISA developed by the present inventors according to the following description:

96-well plates (Costar) were sensitized with the capture antibody (2 μg/ml purified rabbit IgG anti-humanGal1, Natocor) diluted in sensitizing buffer (0.1 M Na₂CO₃ pH 9.5) overnight at 4° C. Then, the plates were washed 3 times for 5 minutes with washing buffer (PBS pH 7.4; Tween-20 0.01% v/v) and were incubated with blocking buffer (PBS; BSA 2% w/v) for 1 hour at room temperature. Subsequently, the samples and the standard recombinant human Gal1 were incubated, detection range 160-2.5 ng/ml, in 100 μl dilution buffer (PBS; BSA 1% w/v) for 18 hours at 4° C., followed by 4 washes for 5 minutes. Once the washes were finished, the detection solution containing the biotinylated secondary antibody (100 ng/ml purified rabbit IgG anti-humanGal1, Natocor) was added for 1 hour followed by streptavidin-HRP solution (0.33 μg/ml; Sigma) for 30 minutes at room temperature. Finally, 3 washes were performed, and detection was carried out with a TMB substrate solution (0.1 mg/ml tetramethylbenzidine) and H₂O₂ 0.06% v/v in citrate-phosphate buffer (0.1M citric acid; 0.1M Na₂PO₄). Reaction was stopped by addition of 50 μl of 2N H₂SO₄ and the absorbance was determined at a wavelength of 450 nm and 550 nm (correction factor) in a Multiskan MS plate spectrophotometer microplate reader (Thermo Electron Corporation).

The amount of Gal1 present in the extracts was relativized in different ways: in the bacterial extracts it was relativized to the amount of total protein; in turn, for conditioned media, the amount of Gal1 present was expressed by ml of culture by OD₆₀₀ unit.

Purification of Gal1 and Variants from Lactococcus lactis NZ9000 Conditioned Media

The optimized L. lactis p170-usp:Gal1 strain generated according to the present invention, was cultured ON in M17G media with chloramphenicol 5 μg/ml at 30° C. without shaking. The saturated culture was diluted 1/10 in the same fresh media and incubation was continued for 5 hours (for obtaining a good amount of Gal1 in the conditioned media).

After centrifugation of the conditioned media at 10000×g for 10 minutes for separation of bacteria (and cellular debris), media was diluted 1/10 with PBS (to a final concentration of PBS 1×, pH 7.4), and produced Gal1 was purified by affinity chromatography on a Lactosyl-Sepharose column as previously described (Pace et al., 2003). After washing the column with PBS containing 4 mM β-mercaptoethanol for removing those macromolecules not bound to the Lactosyl-Sepharose matrix, elution was carried out with lactose buffer (PBS, 4 mM β-mercaptoethanol, 150 mM lactose) drawing (0-12) 600 μl fractions.

Western Blot of Gal1 and Variants from Lactococcus lactis NZ9000 Conditioned Media

From fractions obtained after purification, 32 μl of each fraction were prepared with 8 μl in culture buffer 5× for being separated by SDS-PAGE. The presence of human Gal1 and variants was determined as described above for the protein extracts.

### Scratch Assay

Cells were grown for 24 hs on DMEM medium with 10% fetal bovine serum (FBS) on 24-wells tissue plates. After 24 hours (cell confluence reached ˜80%), two perpendicular scratches were made using 1 ml pipette tips and the wells were washed twice with medium. Cells were allowed to grow for additional 48 hs in DMEM 10% FBS with or without rGal1 (1 μM), TNF (50 μg/ml) and Lactococcus lactis pNZ or Lactococcus lactis p170-usp:Gal1-Opt supernatant (final dilution 1/12.5). Then cells were fixed and stained with 1% crystal violet in 2% methanol. Photographs were taken at 10× magnifications with an Olympus CX31 microscope. Wounded area measured in pixels in each condition, at least 4 fields were analyzed for each assay-triplicate. Area of the wound was measured using ImageJ software and the Wound healing size tool (Suarez-Arnedo et al, 2020).

### Assays for the In Vivo Experimental Model

Preparation of Protein Extracts from Colon

After animal sacrifice by cervical dislocation, three fragments were taken from the small intestine (proximal to the stomach, D; median zone, Y; proximal to the cecum, I) and a fragment from the colon, all of which were processed for total protein extraction and quantitation, as described below:

Colon fragments were disrupted mechanically in protein extraction buffer (50 mM Tris-HCl pH 7.5; 150 mM NaCl; 10 mM EDTA and 1% NP-40) containing protease inhibitor cocktail (Sigma). Lysate was centrifuged at 12000×g for 10 minutes at 4° C. Protein concentration was determined on the supernatant obtained (total protein extract), using the Micro BCA Kit (Pierce) following the provider's instructions. Protein concentration was determined by measuring the absorbance at a wavelength of 550 nm in a plate spectrophotometer (Labsystems Multiskan), following the same procedure with a bovine serum albumin (BSA) standard curve (0.5-100 μg/ml).

### Determination of Gal1

Concentration of Gal1 present in the protein extracts obtained from colon, was determined by ELISA developed in the research lab of the present inventors, previously described. The amount of Gal1 was relativized to the amount of total protein quantified in the extracts.

RNA Extraction from Colon Samples and Real-Time PCR

After animal sacrifice by cervical dislocation, colon fragments were processed for total RNA extraction using TRIzol™ (Thermo Fisher Scientific), without the addition of glycogen, according to the manufacturer's instructions.

RNA obtained was solubilized in RNAse-free water, quantified and quality-checked by NanoDrop (Thermo Fisher Scientific). Then, 2.5 μg of total RNA were treated with 1 Unit of DNAse I (Amplification Grade, Thermo Fisher Scientific) for 15 minutes at r.t. in a suitable buffer, removing eventual genomic-DNA contamination. Reaction was quenched by addition of EDTA at a final concentration of 2.5 mM, and DNAse was inactivated at 50° C. for 15 minutes.

cDNA was obtained from 2.5 μg RNA (pre-treated with DNAse I) using either SuperScript™ II Reverse Transcriptase or SuperScript™ IV Reverse Transcriptase (Thermo Fisher Scientific) following the manufacturer's instructions, in the presence of dideoxynucleotide hexamers at random (random primers, commercially available from Invitrogen™ (Cat No. 48190-011), at a concentration of 2.5 μg/ml), triphosphate dideoxynucleotides (ddNTPs, 500 nM), and 20 Units of RNAseOUT™ Recombinant Ribonuclease Inhibitor (Thermo Fisher Scientific).

From obtained cDNA, a real-time PCR was performed using either SYBR™ Green PCR Master Mix (Thermo Fisher Scientific) or the SsoAdvanced Universal SYBR Green Supermix (Bio-Rad) in the presence of primer pairs for specific dideoxynucleotides for each gene (primers, final concentration of 300 nM each), with a standard cycling protocol in the CFX96 Touch™ Real-Time PCR Detection System (Bio-Rad). The sequences of primers used for each murine gene are shown in Table 1 below.

For determining the level of each gene, a relative quantitation method was performed by a sequence-specific standard curve, relative to the amount of enzyme transcript Glyceraldehide-3-phosphate dehydrogenase (GAPDH) in each sample.

Specific primer pairs used for each galectin are depicted in Table 1 below.

TABLE 1
Nucleotide sequence of primers used.
F: forward primer (5′ to 3′); R: reverse primer (3′ to 5′).

| Gene | Primer 1 (F) (5′-3′) | Primer 2 (R) (5′-3′) |
|---|---|---|
| Galectin-1 | TCAGCCTGGTCAAAGGTGAT | TGAACCTGGGAAAAGACAGC |
| | (SEQ ID NO: 7) | (SEQ ID NO: 8) |
| Interferon gamma | GGCTGTTACTGCCACGGCACA | CACCATCCTTTTGCCAGTTCCTCCA |
| (IFN-γ) | (SEQ ID NO: 9) | (SEQ ID NO: 10) |
| Interleukin 5 (IL-5) | ACACAGCTGTCCGCTCACCG | TCCACAGTACCCCCACGGACA |
| | (SEQ ID NO: 11) | (SEQ ID NO: 12) |
| Interleukin 10 (IL-10) | TTCCCTGGGTGAGAAGCTGA | CTTCACCTGCTCCACTGCCT |
| | (SEQ ID NO: 13) | (SEQ ID NO: 14) |
| Interleukin 17 (IL-17) | CTCCAGAAGGCCCTCAGACTAC | AGCTTTCCCTCCGCATTGACACAG |
| | (SEQ ID NO: 15) | (SEQ ID NO: 16) |
| GAPDH | CAGAACATCATCCCTGCAT | GTTCAGCTCTGGGATGACCTT |
| | (SEQ ID NO: 17) | (SEQ ID NO: 18) |

All primers were purchased from Integrated DNA Technologies (IDT).

Leukocyte Enrichment from Mesenteric Lymph Nodes and Lamina propria of the Colon.

Mice were sacrificed and the mesenteric lymph nodes (MLN) were removed and disaggregated in complete RPMI 1640 culture medium supplemented with 10% v/v FBS (Sigma). This suspension was placed on ice until use. The colon was removed, its contents were removed by thoroughly flushing the colon with cold HBSS buffer (Hank's Balanced Salt Solution) without Ca²⁺/Mg²⁺, then the colon was opened longitudinally and further washed. The tissue was cut into pieces of 5 millimeters long and they were incubated in HBSS buffer without Ca²⁺/Mg²⁺ with EDTA (5 mM) and 5% v/v FBS (Sigma) (37° C., 20 minutes, 120 rpm), to separate the epithelium from the rest of the intestinal tissue. At the end of the incubation, the samples were shaken for 5 seconds in a vortex and passed through a filter of 100 μm pore diameter, the eluate was discarded and the pieces of tissue that remained on the filter were collected to repeat the previous incubation twice, once with EDTA and the last without EDTA. The samples were then transferred to C tubes (MACS) with digestion solution: PBS; FBS 10% v/v (Sigma); Collagenase D 1.5 mg/ml (Roche) and DNAse I 0.1 mg/ml (Thermo Fisher Scientific) (37° C., 60 minutes, 180 rpm). At the end of the enzymatic treatment, samples were disaggregated with gentle MACS™ Dissociator (MACS) and the m_intestine_01 program. Finally, the samples were resuspended in cold PBS buffer and filtered through a 40 μm filter. The resulting cell suspensions were washed once with PBS buffer and centrifuged (300×g, 10 minutes). Cells were resuspended in PBS with 2% FBS and stained for cytometry.

### Flow Cytometry

Flow cytometry was performed using LSRFortessa cytometer (Becton Dickinson), and data were analyzed with FlowJo software (V.10.8.1; FlowJo LLC). Leukocytes from mesenteric lymph nodes and colonic Lamina propria (LP) were stained for flow cytometry analysis as follows: First, cells were incubated with Zombie Aqua™ (BioLegend) following manufacturer instructions. Then cells were stained for 30 min at 4° C. with combinations of the following antibodies in PBS 2% FBS (Sigma):

TABLE 2
Antibodies used in flow cytometry experiments

| | |
|---|---|
| CD45 | PerCP-Cy5.5 |
| CD138 | BV421 |
| B220 | BV711 |
| CD19 | APC-FIRE750 |
| CD3 | APC |
| CD4 | AF700 |
| CD8 | FITC |
| CD69 | PE-CY7 |
| Ly6C | BV650 |
| Ly6G | PECy7 |
| MHCII | AF488 |
| CD64 | BV421 |
| Siglec-F | APC |
| CD11c | APC-Cy7 |
| CD11b | BV605 |

Finally, cells were fixed with 1% formaldehyde for 30 min and saved in PBS 2% FBS until data acquisition.

### EXAMPLES

The invention will now be further described based on the following examples. It is to be understood that these examples are intended for illustrative purposes only, and by no means should be construed to be limiting the scope of the invention, which is only defined by the appended claims.

### Example 1-Preparation of L. lactis Strains Expressing Gal1

Considering the immunoregulatory activity of Gal1 and seeking for a targeted and selective delivery of this potential therapeutic (to the intestinal system/to inflamed tissue), the present inventors optimized the expression of rGal1 in Lactococcus lactis subsp. cremoris NZ9000.

For the generation of Lactococcus lactis strains, human recombinant Gal1 was expressed in Lactococcus lactis subsp. cremoris NZ9000 (genotype pepN::nisRnisK). This strain contains the regulatory genes nisR and nisK integrated to the genome within the pepN gene, which turns it into a standard strain for regulation of genic expression by the cyclic peptide nisin (NICE® system, NIsin Controlled gene Expression system).

This strain was transformed by electroporation with different constructs of the human Gal1 gene (LGALS1) into the plasmid pNZ8048 (without Histidine tag, Histag), containing the PnisA promoter (pNZ), for expressing heterologous proteins by addition of nisin, and also confers resistance to chloramphenicol antibiotic, as a selection system.

Bacteria were transformed by electroporation with plasmid vector pNZ8048 containing human Gal1 cDNA sequence under the control of a promoter inducible by cyclic peptide nisin.

The following strains of the Lactococcus lactis NZ9000 were thus obtained: a) L. lactis pNZ, transformed with the empty plasmid pNZ8048, used as control; and b) L. lactis pNZ-Gal1, transformed with said plasmid containing the codifying sequence of human Gal1.

For the production of human Galectin-1 in L. lactis NZ9000 through the NICE® system, the different bacterial strains generated by transformation were cultured ON in M17G medium with chloramphenicol 5 μg/ml at 30° C. with no shaking. Different induction times were assayed. The saturated culture was diluted to an OD₆₀₀ of 0.08 in the same fresh medium, and incubation was continued until an OD₆₀₀ of 0.6-0.8 was reached.

After addition of nisin (20 ng/ml) for induction of the expression of the genes cloned under the control of the PnisA promoter, the cells were cultured at different times at 30° C., without shaking.

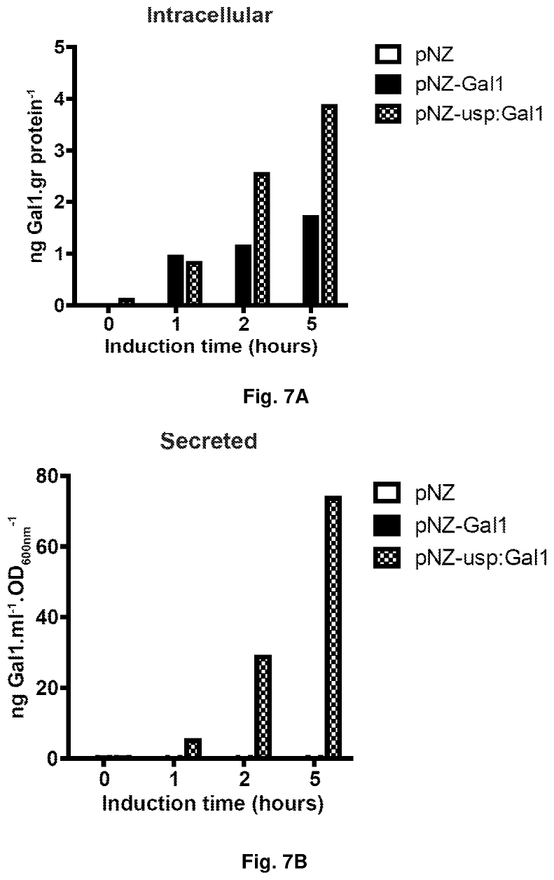

ELISA assays demonstrated the presence of rGal1 in the intracellular fraction, but production of this heterologous protein by the genetically modified L. lactis pNZ-Gal1 was limited by cytosolic accumulation of protein (FIG. 7A).

### Example 2-Preparation of L. lactis Strain Expressing Gal1 (Extracellular)

In order to overcome the above-mentioned limitation in the production of the heterologous protein, a third strain of genetically engineered L. lactis capable of secreting rGal1 (L. lactis pNZ-usp:Gal1) was generated, where the expression cassette included a secretion signal (Usp45) fused to the 5′ end of human Gal1 sequence. Usp45 is a short amino acid sequence which will be recognized by the bacterial secretory system, and as a consequence the protein is released into the culture media (in vitro production) or into the gastrointestinal lumen (in vivo assays) after cleavage of Usp45.

For the production of human Galectin-1 in L. lactis NZ9000 using the NICE® system, different bacterial strains generated by transformation were cultured overnight (ON) in M17G with chloramphenicol 5 μg/ml at 30° C. with no shaking. The saturated culture was diluted to anOD₆₀₀ of 0.08 in the same fresh medium, and incubation was continued until anOD₆₀₀ of 0.6-0.8 was reached. After the addition of nisin (20 ng/ml) for inducing expression of the genes cloned under the control of the PnisA promoter, cells were cultured at different times at 30° C., with no shaking.

L. lactis pNZ-usp:Gal1 produced higher amounts of intracellular rGal1 (FIG. 7A) and secretion levels of this heterologous protein that were proportional to the induction time as demonstrated both by ELISA (FIG. 7B), and Western blot (FIG. 8).

It was observed that rGal1 expression in L. lactis pNZ-Gal1 and L. lactis pNZ-usp:Gal1 was efficient, but the levels obtained were too low for maintaining lectin activity. Conditioned media from L. lactis pNZ-usp:Gal1 contained approximately 100 ng/ml of this lectin (˜7 nM), and considering that rGal1 activity is dependent on its dimeric form (dimerization constant 7 μM), the possibility that although efficiently expressed, rGal1 obtained from L. lactis may not be functional, was considered. To evaluate ligand recognition, rGal1 binding both from intracellular fractions and conditioned media from L. lactis pNZ-Gal1 and L. lactis pNZ-usp:Gal1 was tested using a lactosyl-Sepharose column. Results showed no binding to the canonical ligand, lactose.

### Example 3-TNBS-Induced Colitis Model in Mice with L. lactis pNZ-Usp:Gal1

Finally, and considering that rGal1 administered intravenously (Santucci et al., 2003) or intraperitoneally (Morosi et al., 2021) can reduce the symptoms of TNBS-induced intestinal inflammation in mice, the therapeutic potential of L. lactis pNZ-usp:Gal1 was tested in a TNBS model of colitis.

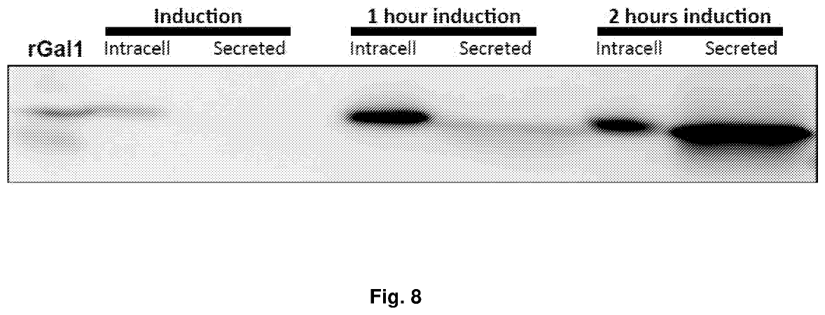

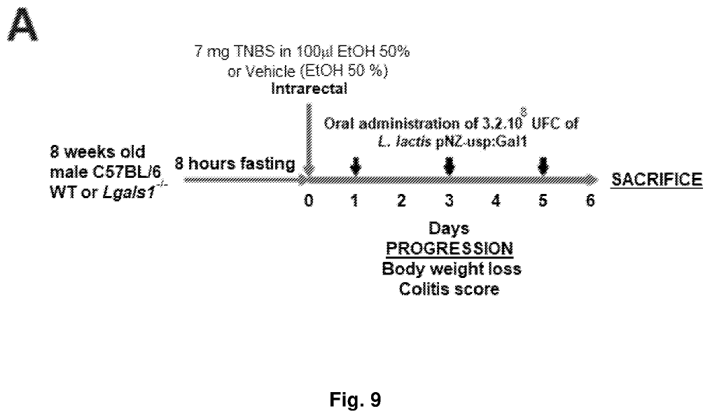

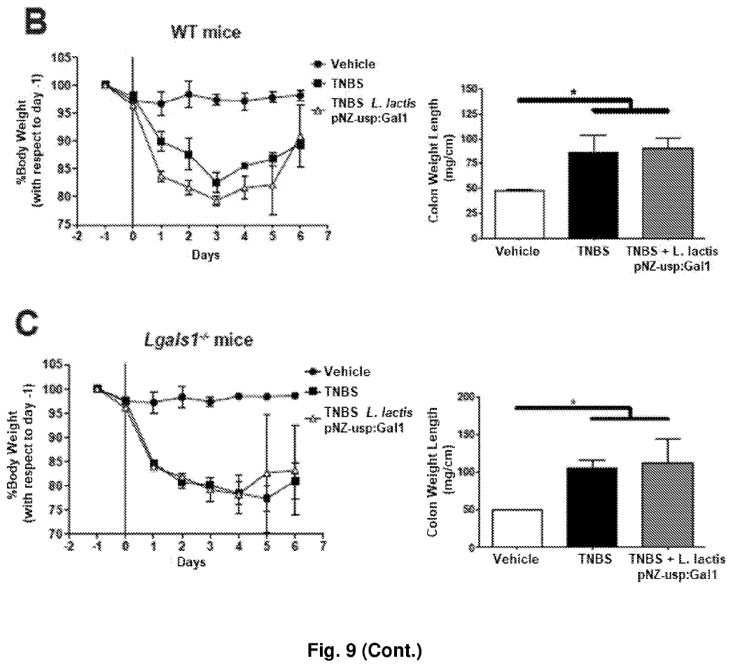

The therapeutic administration protocol of human Gal1-producing L. lactis in TNBS-induced acute colitis is outlined in FIG. 9A.

8 weeks old wild-type (WT) or Gal1-deficient (Lgals1^{-/-}) C57BL/6 male mice were used for inducing experimental colitis by TNBS instillation. Mice were subject to fasting for 8 hours until colon was emptied. Subsequently, they were instilled with 100 μl of a TNBS solution in ethanol 50% v/v (from a 1 M TNBS aq. solution, Sigma) intrarectally or 100 μl of Vehicle solution (ethanol 50% v/v), using an i.v. 20G Teflon catheter (Dexal), approaching at 3-4 cm from the rectus. The volume during instillation was slowly released and smoothly removing the catheter so that colon was exposed to the TNBS solution as much as possible. Upon complete removal of the catheter, mice were hold in an inclined position for around 10 seconds (head downwards) for avoiding loss of TNBS solution. The TNBS dose administered was 7 mg per animal. Body weight of the animals was measured daily for determining the loss percentage caused by colitis (wasting disease) (Fuss et al., 1999; Wirtz et al., 2017). The weight of animals on the day before fasting (day−1) was taken as the starting weight. Six days after instillation were sacrificed by cervical dislocation and the tissues of interest were removed for further analysis.

TNBS-treated animals were separated into 2 groups: one group received 3.2×10⁸ CFU of the L. lactis pNZ-usp:Gal1 strain in 100 μl of physiologic solution, through oral route (using a 20G gastric probe), every two days (on days 1, 3 and 5) (3 administrations in total). The other group received 100 μl of physiologic solution, through oral route and with a similar regime than the L. lactis-treated group.

In addition to the previous results showing no ligand recognition, there was no improvement of inflammation in the TNBS-treated mice (FIGS. 9B and 9C), indicating that rGal1 produced by L. lactis may not be functional at a biochemical or biological level probably due to the low concentrations obtained.

### Example 4-Preparation of an Optimized L. lactis Strain for Expression of Gal1 and Variants

With the purpose of increasing the expression of human Gal1 in L. lactis, codons for full-length usp45: Gal1 gene were optimized for expression in this bacterium (GeneScript). In order to become independent from the nisin addition, promoter PnisA was replaced with the P170 promoter (p170), which is inducible by acidic pH. The total construct was synthesized (Integrated DNA Technologies, IDT), and inserted into the plasmid pNZ8048 (without Histidine tag). Thus, the strain optimized L. lactis p170-usp:Gal1 was generated.

To overcome these limitations, it was decided to proceed as follows: i) optimizing codons for L. lactis expression, ii) changing the promoter in the expression plasmid, as nisin showed high variability between experiments. Induction by acidic pH (p170-usp:Gal1) was used. Codon optimization for expression and secretion in the bacterial system of the invention was carried out as described below.

Nucleotide sequences: Codon optimization was done only in Gal1/SGs sequences. P170 and Usp45 sequences were not altered (these do not require optimization).

The following protocol was carried out for the design of the p170 promoter region:

From the NCBI sequence with accession number AJ011913 (Madsen et al. 1999) the region −148 to +4 was used (with respect to the transcriptional start site, +1). Next to it, the region +77 to +115 was added. Then the “cgggtctaaattagggta” nucleotide sequence (spacer sequence of SEQ ID NO: 21) from the pAK80 plasmid was included to properly separate the promoter and RBS (ribosome binding site). PstI restriction site was included. Next to it, the RBS from pNZ8048 plasmid followed by the NdeI restriction site were added.

The following protocol was carried out for the design of the Gal1 variants sequences:

To obtain the Gal1 variants (SGs) optimized for expression in L. lactis, the sequence of optimized Gal1 was used as template. Then, the required mutations were introduced in the sequence using the most frequent L. lactis codon for replacement of the wild type codons.

### DNA Synthesis

P170-usp-gal1:

A DNA fragment synthesis was ordered to Genscript containing the P170 promoter region, together with the secretion signal Usp45 and Gal1 coding sequence; codon optimization for expression in L. lactis was restricted only to Gal1 sequence. The DNA fragment was delivered in the pUC57-kan plasmid (pUC57-kan-P170-usp-gal1).

P170 sequence (SEQ ID NO: 19):
aagcttagatctggaagtaattctagagctttgcatgtctataaaaaaa
tgggattccaaattaccgatatcaatatgcgaaaagaactatgaatatc
cactccatttttggttgccatttgttaacgctgcctcctctccctagtg
ctataataaaaatggcccattttggaacagacttctactattttgttgt
ctagtagcgggtctaaattagggtactgcagaaggaggtattc
Usp45 sequence (SEQ ID NO: 20):
atgaaaaaaaagattatctcagctattttaatgtctacagtgatacttt
cagcagcagccccgttgtcaggtgtttacgctgcc

### Gal1 Variants (SGs):

DNA fragments containing the SGs were ordered to Genscript. The DNA fragments were delivered in the pUC57-simple plasmid.

### Gal1 Cloning

To express the optimized Gal1 in lactic acid bacteria, pNZ8048 and pUC57-kan-P170-usp-gal1 plasmids were digested with BgIII and HindIII enzymes (Thermofisher), DNA fragments were separated in agarose gels, recovered and purified using a commercial kit (Wizard® SV Gel and PCR Clean-Up System, Promega) following manufacturer's instructions. Next, digested pNZ8048 plasmid and Gal1 fragment were ligated using T4 DNA ligase (Invitrogen). Ligation mixture was desalted and electroporated in L. lactis NZ9000 electrocompetent cells. Isolated colonies were obtained in M17 medium+glucose 0.5% w/v+0.5M sucrose using chloramphenicol 10 μg/ml as selection antibiotic. Presence of P170-usp-gal1 was detected by colony PCR using specific primers that hybridize flanking the cloned insert. Correct cloning was confirmed by Sanger sequencing, naming the obtained plasmid as pNZ170-usp-gal1.

### Cloning of the Gal1 Variants

Plasmids pNZ170-usp-gal1 and pUC57-Simple containing the different SGs were digested with NcoI and HindIII enzymes (Thermofisher). DNA fragments were separated in agarose gels; pNZ170-usp, SG1, SG2, SG3 and SG4 were recovered and purified using the commercial kit describe previously. The pNZ170-usp vector was ligated with each of the SGs and continued as described previously, correct cloning was confirmed by Sanger sequencing. The plasmids were named pNZ170-usp-SG1, pNZ170-usp-SG2, pNZ170-usp-SG3 and pNZ170-usp-SG4.

Codon optimization improved the efficiency of protein expression, and in the case of rGal1 the optimization was performed using a sophisticated algorithm that involved a variety of parameters critical for transcription, translation and protein folding. As a result of applying the algorithm 30% of the nucleotides of rGal1 were changed. Thus, a much more efficient expression of the protein was obtained.

# Algorithm: Global alignment
# Length of sequence 1: 528 nt
# Length of sequence 2: 408 nt
# Similarity: 287/408 (70.34%)
# Number of mismatched: 121 nt
Sequence for WT human Galectin-1 (Seq ID No 6):
ATCTCTCTCGGGTGGAGTCTTCTGACAGCTGGTGCGCCTGCCCGGGAACATCCTC
CTGGACTCAATCATGGCTTGTGGTCTGGTCGCCAGCAACCTGAATCTCAAACCTG
GAGAGTGCCTTCGAGTGCGAGGCGAGGTGGCTCCTGACGCTAAGAGCTTCGTGC
TGAACCTGGGCAAAGACAGCAACAACCTGTGCCTGCACTTCAACCCTCGOTTCAA
CGCCCACGGCGACGCCAACACCATCGTGTGCAACAGCAAGGACGGCGGGGCCTG
GGGGACCGAGCAGCGGGAGGCTGTCTTTCCCTTCCAGCCTGGAAGTGTTGCAGA
GGTGTGCATCACCTTCGACCAGGCCAACCTGACCGTCAAGCTGCCAGATGGATAC
GAATTCAAGTTCCCCAACCGCCTCAACCTGGAGGCCATCAACTACATGGCAGCTG
ACGGTGACTTCAAGATCAAATGTGTGGCCTTTGACTGAAATCAGCCAGCCCATGGC
CCCCAATAAAGGCAGCTGCCTCTGCTCCCTCTGAA
Sequence for optimized WT Gal1
(Seq ID No 1)
--------------------------------------
ATGGCTTGTGGTCTTGTTGCTAGTAATCTTAATTTGAAACCTGGTGAATGTTTGCGA
GTTCGTGGTGAAGTTGCTCCTGATGCTAAATCATTTGTTCTTAATCTTGGTAAAGAT
TCAAATAATTTGTGTCTTCATTTTAATCCACGTTTTAATGCTCATGGAGATGCAAATA
CAATTGTTTGTAATTCAAAAGATGGTGGAGCTTGGGGTACTGAACAAAGAGAAGCA
GTATTTCCATTTCAACCTGGATCTGTTGCTGAAGTATGTATTACATTTGATCAAGCA
AATCTTACTGTAAAACTTCCAGATGGTTATGAATTTAAATTTCCTAATCGACTTAATT
TGGAAGCTATTAATTATATGGCTGCTGATGGAGATTTTAAAATTAAATGTGTTGCTT
TTGATTAA--------------------------------

The nucleotide sequences codifying the optimized SuperGals of the invention (SG1-SG4) are as follows:

SG1 opt
(SEQ ID NO: 2)
ccatggcttcaggtcttgttgctagtaatcttaatttgaaacctggtga
atcattgcgagttcgtggtgaagttgctcctgatgctaaatcatttgtt
cttaatcttggtaaagattcaaataatttgtgtcttcattttaatccac
gttttaatgctaatggagatgcaaatacaattgtttgtaattcaaaaga
tggtggagcttggggtactgaacaaagagaagcagtatttccatttcaa
cctggatctgttgctgaagtatgtattacatttgatcaagcaaatctta
ctgtaaaacttccagatggttatgaatttaaatttcctaatcgacttaa
tttggaagctattaattatatggctgctgatggagattttaaaattaaa
tgtgttgcttttgattaaggtaccaagctt
SG2 opt
(SEQ ID NO: 3)
ccatggcttcaggtcttgttgctagtaatcttaatttgaaacctggtga
atcattgcgagttcgtggtgaagttgctcctgatgctaaatcatttgtt
cttaatcttggtaaagattcaaataatttgtgtcttcattttaatccac
gttttaatgcttatggagatgcaaatacaattgtttgtaattcaaaaga
tggtggagcttggggtactgaacaaagagaagcagtatttccatttcaa
cctggatctgttgctgaagtatgtattacatttgatcaagcaaatctta
ctgtaaaacttccagatggttatgaatttaaatttcctaatcgacttaa
tttggaagctattaattatatggctgctgatggagattttaaaattaaa
tgtgttgcttttgattaaggtaccaagctt
SG3 opt
(SEQ ID NO: 4)
ccatggcttcaggtcttgttgctagtaatcttaatttgaaacctggtga
atgtttgcgagttcgtggtgaagttgctcctgatgctaaatcatttgtt
cttaatcttggtaaagattcaaataatttgtgtcttcattttaatccac
gttttaatgctaatggagatgcaaatacaattgtttgtaattcaaaaga
tggtggagcttggggtactgaacaaagagaagcagtatttccatttcaa
cctggatctgttgctgaagtatcaattacatttgatcaagcaaatctta
ctgtaaaacttccagatggttatgaatttaaatttcctaatcgacttaa
tttggaagctattaattatatggctgctgatggagattttaaaattaaa
tgtgttgcttttgattaaggtaccaagctt
SG4 opt
(SEQ ID NO: 5)
ccatggcttcaggtcttgttgctagtaatcttaatttgaaacctggtga
atgtttgcgagttcgtggtgaagttgctcctgatgctaaatcatttgtt
cttaatcttggtaaagattcaaataatttgtgtcttcattttaatccac
gttttaatgcttatggagatgcaaatacaattgtttgtaattcaaaaga
tggtggagcttggggtactgaacaaagagaagcagtatttccatttcaa
cctggatctgttgctgaagtatcaattacatttgatcaagcaaatctta
ctgtaaaacttccagatggttatgaatttaaatttcctaatcgacttaa
tttggaagctattaattatatggctgctgatggagattttaaaattaaa
tgtgttgcttttgattaaggtaccaagctt

The optimized L. lactis p170-usp:Gal1 strain was cultured overnight (ON) in M17G media with chloramphenicol 5 μg/ml at 30° C. without shaking. The saturated culture was diluted to an OD₆₀₀ of 0.08 in the same fresh medium, and incubation was continued for another 3 to 5 hours. The medium acidification itself-due to the bacterial growth-induces P170.

As can be seen in FIG. 10, this strategy was productive: the amounts of intracellular rGal1 were considerably higher both in codon-optimized pNZ-usp:Gal1-Opt and p170-usp:Gal1-Opt, while secreted protein in p170-usp:Gal1-Opt was close to 2 μg/ml media (approximately 140 nM, twenty times higher than in L. lactis pNZ-usp:Gal1). The double band observed in FIG. 10 for the intracellular fraction is due to the expression of precursor form usp-Gal1 and secreted Gal1 (without the Usp45 signal). The levels of protein were determined both by Western blot (FIG. 10A) and ELISA and normalized by total protein (FIG. 10B) or optical density (FIG. 10C).

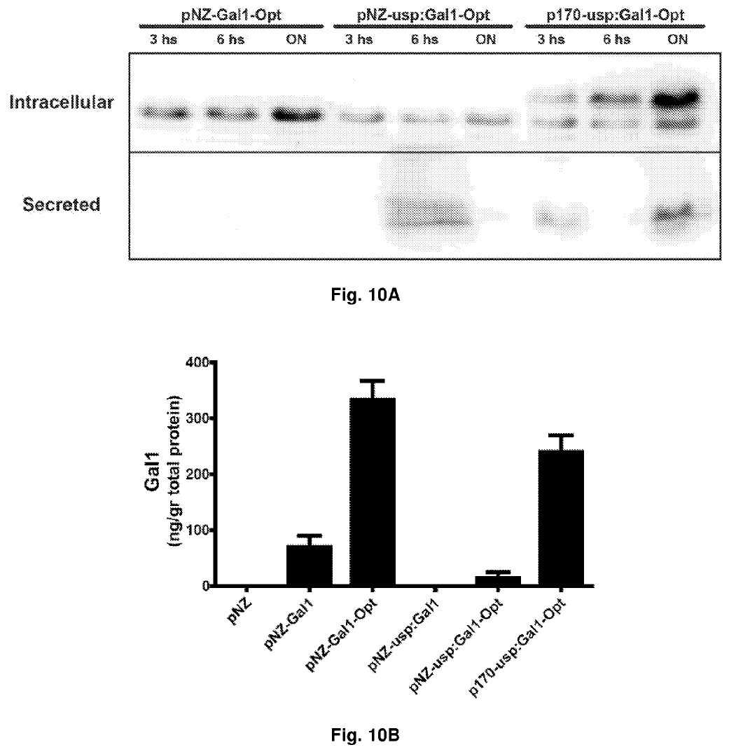

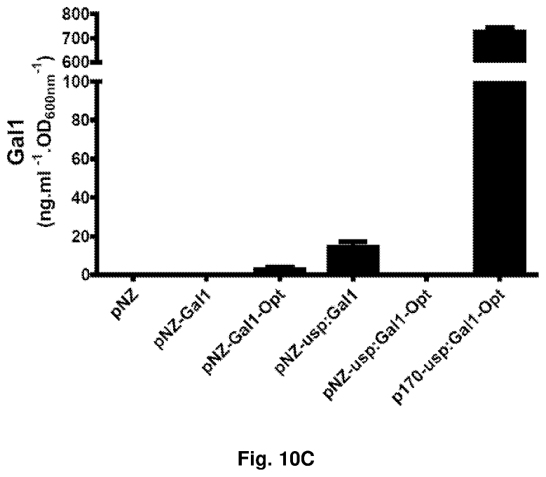

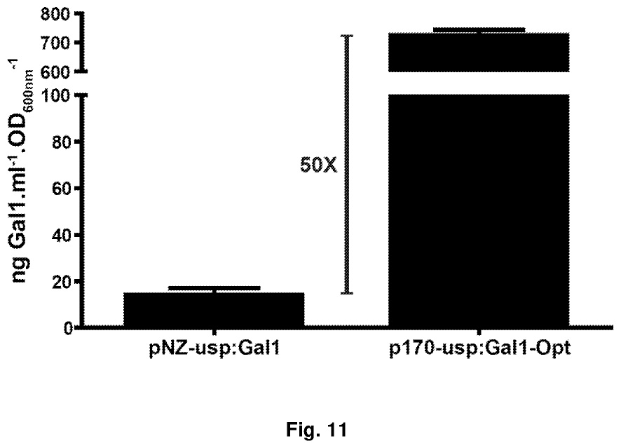

When compared to conditioned media from L. lactis pNZ-usp:Gal1, p170-usp:Gal1-Opt demonstrated 50 times higher levels of rGal1 secretion (FIG. 11), reaching an estimated concentration of 0.35 μM. Although still under Kd, the protein levels were considerably increased and would favour dimerization and biological activity, so it was decided to test rGal1 biochemical and biological properties.

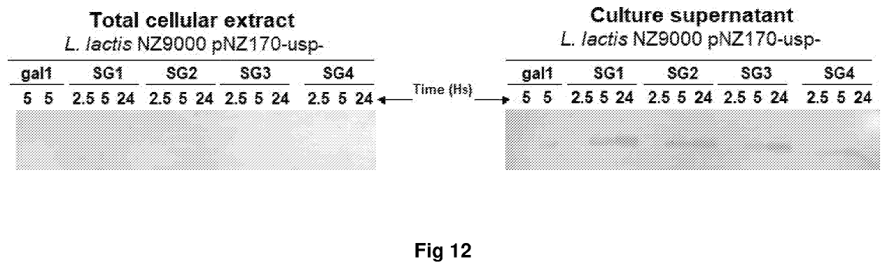

Optimized conditions for Gal1 expression were applied for Gal1 variants (FIG. 12).

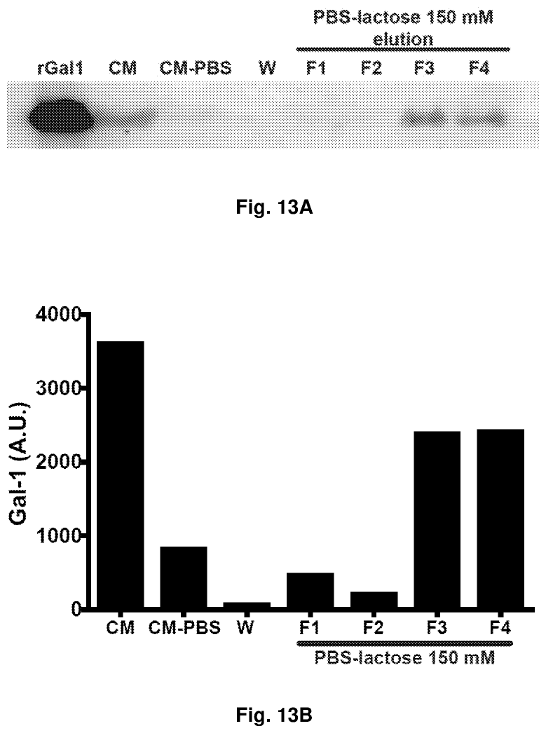

First, a lactosyl-Sepharose column was loaded with conditioned media from a L. lactis p170-usp:Gal1-Opt 5 hour-culture previously diluted in PBS. After several washes with PBS, the rGal1 bound to the canonical ligand was eluted with lactose 150 mM (see materials and methods). The resulting fractions were analyzed for protein content, and Western blot demonstrated that rGal1 was present in the original conditioned media (CM, FIG. 13), and in the elution fractions containing lactose (F3 and F4, FIG. 13A). In opposition to what was observed in the case of L. lactis pNZ-usp:Gal1, rGal1 produced by L. lactis p170-usp:Gal1-Opt maintains its lectin activity (FIG. 13B).

These results show that the newly developed L. lactis NZ9000 strain, optimized to produce and secrete rGal1 under acidic pH, is capable not only to produce the protein but also secretes an active form of rGal1.

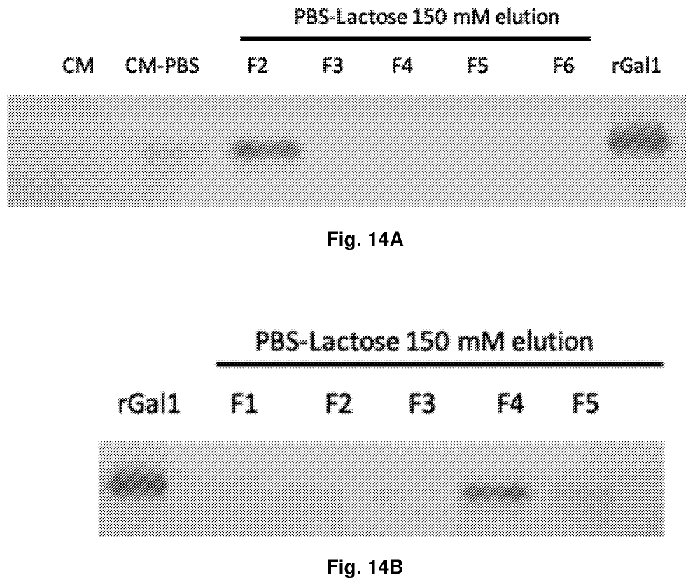

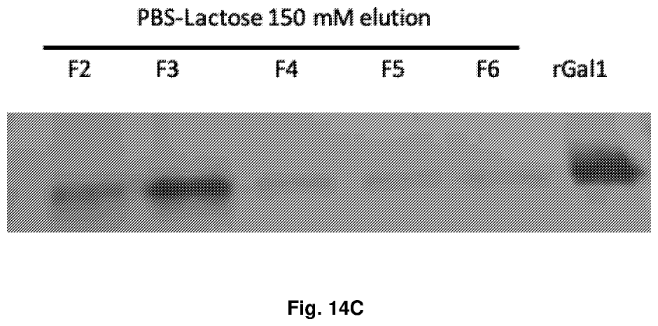

Same conditions for purification provided proof of the activity of Gal1 variants produced in L. lactis (FIG. 14A,B,C).

### Example 5-In Vitro Scratch Assays

Intestinal mucosal wounds are typical in intestinal inflammation, contributing to the breakdown of the intestinal epithelial barrier. As a result, intestinal mucosa counteracts by triggering a wound healing process, which is controlled by many complex mechanisms (Sommer et al., 2021).

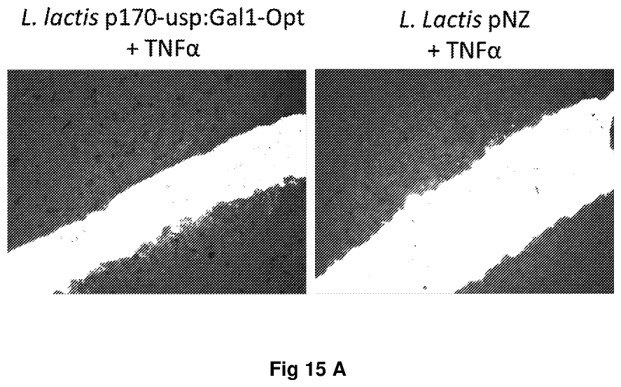

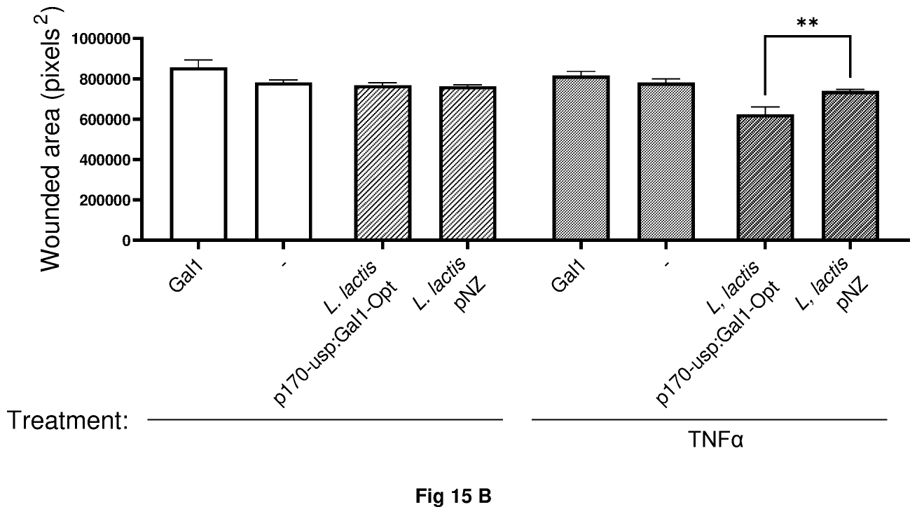

Considering that Gal-2 and Gal-4 have been described as mediators in intestinal wound healing (Paclik et al., 2008), and that Gal-1 binds with greater affinity to intestinal epithelial cells exposed in vitro or in vivo to inflammatory stimuli (Muglia et al., 2016), the present inventors decided to investigate if L. lactis producing Gal1 (L. lactis p170-usp:Gal1-Opt) could promote epithelial wound healing. For this, in vitro scratch assays were performed using the HT29 colon adenocarcinoma cell line as a model. Cells were grown for 24 h on DMEM 10% FBS on 24-wells tissue plates. After 24 h (cell confluence reached ˜80%), two perpendicular scratches were made using 1 ml pipette tips and the wells were washed twice with medium. Cells were allowed to grow for additional 48 h in DMEM 10% FBS with or without rhGal1 (1 mM) and L. lactis-pNZ or L. lactis p170-usp:Gal1-Opt supernatant (5 h culture, diluted 1/12.5). To assess the relevance of Gal1 delivery in an inflammatory context, these experiments were also performed in the presence or absence of TNF (50 μg/ml), a proinflammatory cytokine highly relevant in intestinal inflammation. Finally, cells were fixed and stained with 1% crystal violet in 2% methanol. Photographs were taken at 10× magnifications with an Olympus CX31 microscope. Wounded area measured in pixels in each condition, at least 4 fields were analyzed for each triplicate. Area of the wound was measured using ImageJ software and the Wound healing size tool (Suarez-Arnedo et al., 2020). Results show that supernatant from L. lactis p170-usp:Gal1-Opt is able to reduce the wounded area in a proinflammatory cytokine context, compared with L. lactis pNZ supernatant (FIG. 15A,B). Notably, no reduction in the wounded area is seen with L. lactis (any strain) or rhGal1 without TNF; moreover, rhGal1 is not able to induce would healing in the presence of TNF (FIG. 15B). These results indicate that wound healing by L. lactis pNZ-usp:Gal1 is TNF-dependent, and associated to this specific Gal1 delivery method.

### Example 6-Determination of Immunomodulatory Activity of Gal1-Producing L. lactis Optimized Strains in Mice (Measurement of Inflammation)

To evaluate the immunomodulatory activity of Gal1-producing L. Lactis optimized strains in vivo, colitis was induced by TNBS administration in galectin-1 deficient (Lgals1^{-/-}) mice and WT mice. These animals were treated daily by oral gavage with L. lactis p170-usp:Gal1-Opt, L. lactis p170-usp:SG1-Opt or control strain L. lactis pNZ (1×10⁹ CFU/day).

All bacterial strains (L. lactis p170-usp:Gal1-Opt, L. lactis p170-usp:SG1-Opt, and L. lactis pNZ) were grown ON at 30° C., with no shaking, in M17G medium with 5 μg/ml chloramphenicol. Subsequently, culture was diluted 1/10 in M17G fresh medium (with chloramphenicol) and grown for other 5 hours at 30° C. with no shaking. After this time, OD 600 of the cultures was determined, bacteria were harvested by centrifugation at 10000×g for 5 minutes, washed in sterile physiologic solution and centrifuged again. Finally, bacteria were resuspended in sterile physiologic solution for administration to mice.

The therapeutic administration protocol of human Gal1-producing L. lactis in TNBS-induced experimental colitis is outlined below.

8-12 week-old Gal1-deficient (Lgals1^{-/-}) C57BL/6 male mice were used for inducing experimental colitis by double TNBS instillation. Mice were subject to fasting for 6 h with concomitant ad libitum access to glucose 5% w/v in the drinking water. Subsequently, they were instilled with 100 μl of a TNBS solution in ethanol 50% v/v (from a 1 M TNBS aq. solution, Sigma) intrarectally, using an i.v. 20G Teflon catheter (Dexal), approaching at 3-4 cm from the rectus. The volume during instillation was slowly released and smoothly removing the catheter so that colon was exposed to the TNBS solution as much as possible. Upon complete removal of the catheter, mice were hold in an inclined position for around 10 seconds (head downwards) for avoiding loss of TNBS solution. The TNBS dosage was optimized for this resistant strain and calculated for each animal considering their body weight: 0.15 mg of TNBS per gram of animal weight. Body weight of the animals was measured daily for determining the loss percentage caused by colitis (wasting disease) (Fuss et al., 1999; Wirtz et al., 2017). The weight of animals on the day before fasting (day −1) was taken as the starting weight. Seven days after instillation animals were challenged intrarectally for the second time with TNBS. The TNBS dosage was again calculated based on body weight. Three days after the second instillation (ten days from beginning of the colitis protocol) animals were sacrificed by cervical dislocation and the tissues of interest were removed for further analysis.

TNBS-treated animals of either genotype were separated into 2 groups: one group received 1×10⁹ CFU of the L. lactis pNZ strain daily in 100 μl of saline solution, through oral route (using a 20G gastric probe), from day 0 through day 9 incl. (10 administrations in total). The other group received 1×10⁹ CFU of the optimized L. lactis p170-usp:Gal1 in 100 μl of saline solution, through oral route and with a similar regime than the control group. Same protocol was applied when comparing L. lactis p170-usp:SG1 to the control L. lactis pNZ strain.

Macroscopic inflammation of the colon was assessed by measuring the weight/length ratio after sacrifice.

After animal sacrifice, colon was totally extracted by performing a cut right under the cecum and a cut at the rectum.

Colon was measured with a ruler and weighed in a weighing scale with its contents. From these two measurements, the weight/length ratio of the colon was determined and expressed in mg/cm.

The results shown in FIG. 16A demonstrate that Lgals1^{-/-} mice receiving L. lactis p170-usp:Gal1-Opt had a reduced weight loss (wasting disease) and a decreased colon weight/length ratio (FIG. 16B), both signs of milder intestinal inflammation, when compared to those treated with L. lactis pNZ (empty vector).

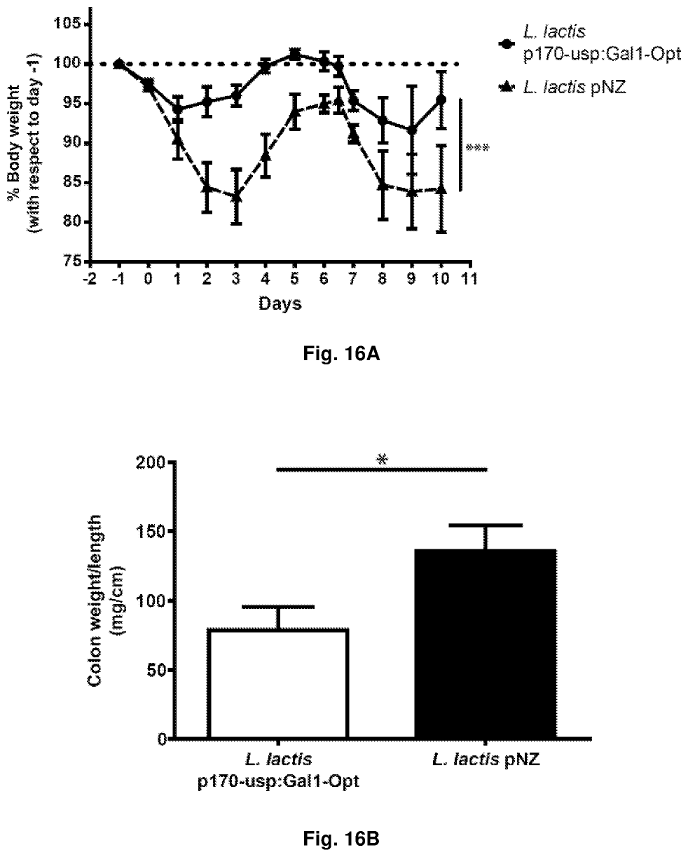

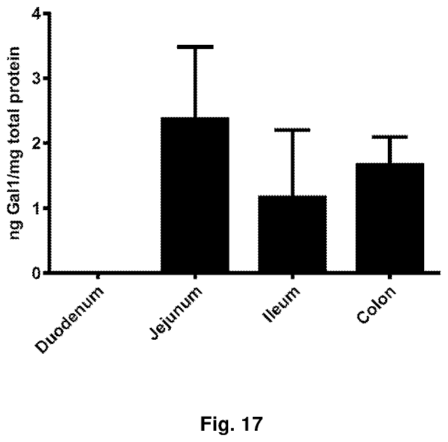

Delivery of rGal1 to different parts of the digestive tract (duodenum, jejunum, ileum, colon) was confirmed by ELISA on Lgals1^{-/-} mice treated with L. lactis p170-usp:Gal1-Opt. Quantification of rGal1 by ELISA was normalized to total protein content in these four intestine regions. As can be seen in FIG. 17, rGal1 is undetectable in duodenum, but detectable at in the jejunum, ileum and colon.

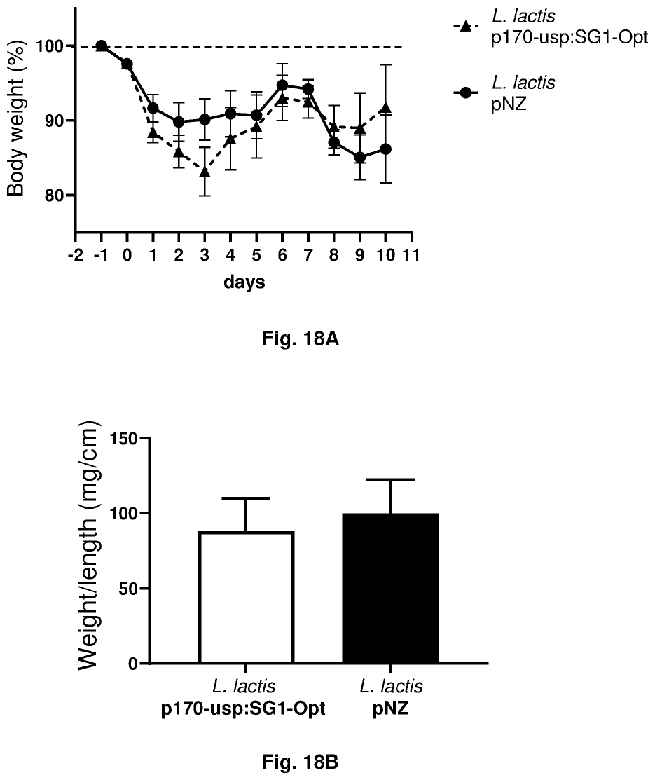

When analyzing daily administration of 1.10⁹ CFU of L. lactis p170-usp:SG1-Opt (bacteria expressing the Gal1 variant SG1) or L. lactis pNZ (control strain) in Lgals1^{-/-} mice coursing colitis, FIGS. 18A and 18B show that animals receiving L. lactis p170-usp:SG1-Opt show less body weight loss after the second TNBS instillation (FIG. 18A) with no significant differences in colon weight/length at sacrifice (18 B).

Considering that Lgals1^{-/-} mice coursing TNBS-induced colitis are a relevant model for understanding the role of Gal1 in intestinal inflammation, but does not mirror human intestinal inflammation (where Gal1 increases its expression in inflamed tissue both in CD and UC, Papa Gobbi et al, 2016; Morosi et al., 2021), the inventors then evaluated daily treatment of 1.10⁹ CFU of L. lactis p170-usp:Gal1-Opt or L. lactis pNZ (control strain) in C57Bl/6 WT mice coursing TNBS-induced colitis. Results show that even though body weight curves (FIG. 19A) and colon weight/length at sacrifice (19B, p=0.0537) are not significantly different between animals that received the control strain or the Gal1-producing L. lactis, animals receiving L. lactis p170-usp:Gal1-Opt show decreased colon weight/length ratio (FIG. 19B).

### Example 7-Immunomodulatory Properties of Gal1-Producing L. lactis Optimized Strain (Evaluation of Immune Cell Landscape)

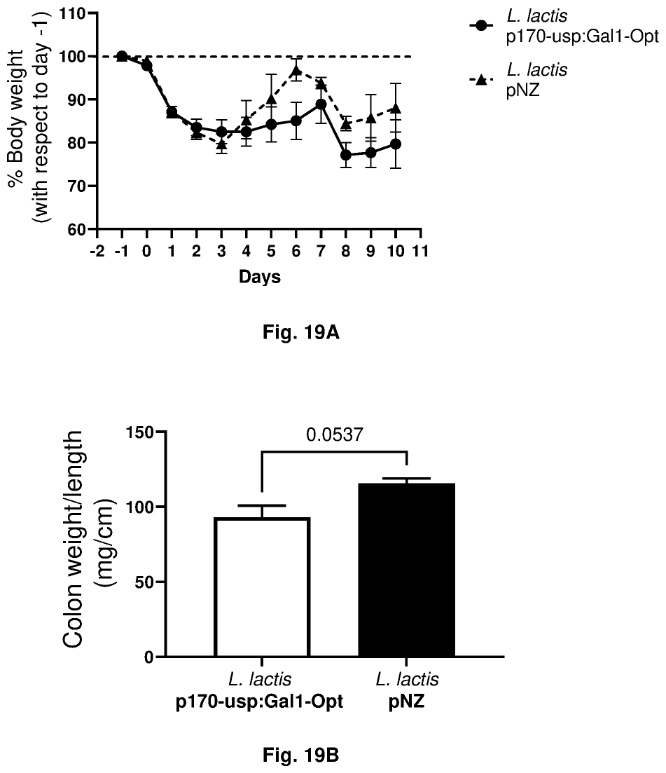

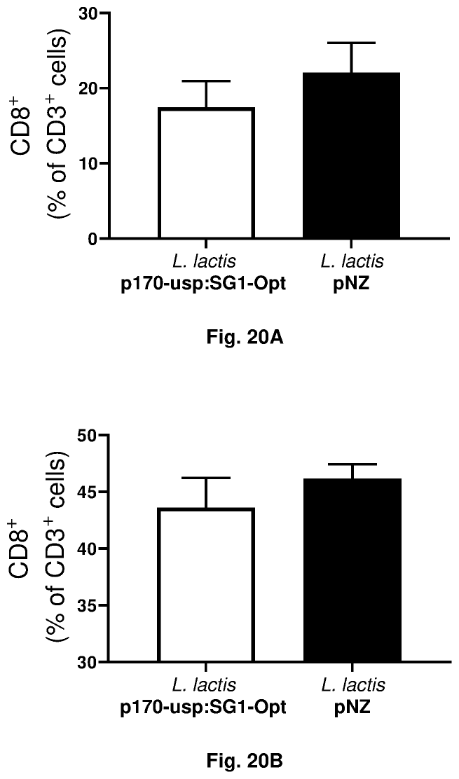

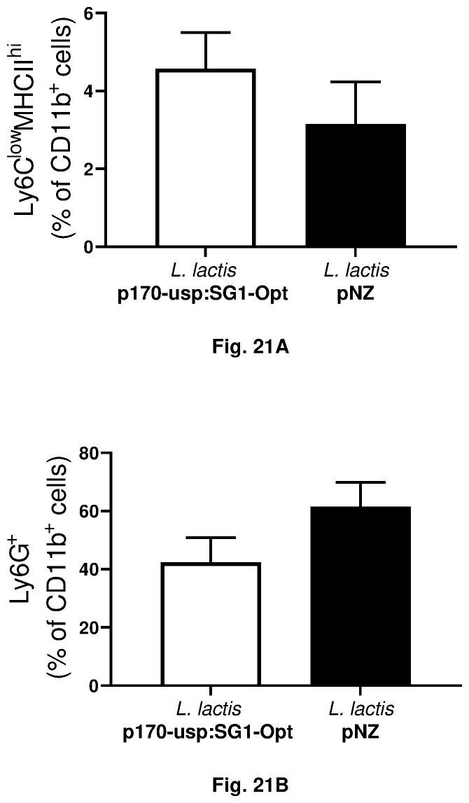

Lgals1^{-/-} mice daily treated with 1.10⁹ CFU of L. lactis p170-usp:SG1-Opt show a tendency towards a decreased proportion of CD8⁺ T cells in Lamina propria (LP) (FIG. 20A) and mesenteric lymph nodes (FIG. 20B) when compared to animals receiving control strain (L. lactis pNZ). In turn, these animals also show an increase in macrophages CD11b⁺Ly6C^{low}MHCII^{hi} (FIG. 21A) and a decrease in neutrophils CD11b⁺Ly6G⁺ (FIG. 21B) in Lamina propria (LP). The former results are in line with previously published work, where i.p. injected Gal1 was able to modulate the CD8⁺CD69⁺ T cell population in Lgals1^{-/-} mice coursing colitis (Morosi et al, 2021).

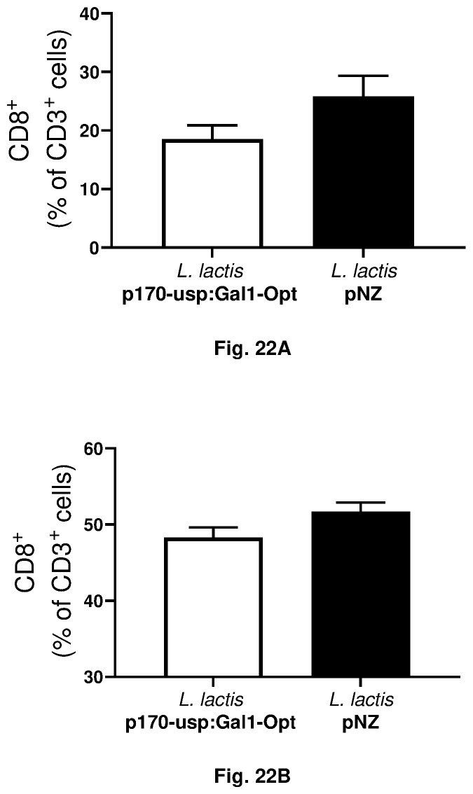

In turn, administration of L. lactis p170-usp:Gal1-Opt in WT animals coursing TNBS-induced colitis also induces a tendency to decrease the CD8⁺ T cell population, key cells mediating inflammation in this colitis model. These results were observed both in Lamina propria (LP) (FIG. 22A) and mesenteric lymph nodes (FIG. 22B).

### Example 8-Immunomodulatory Properties of Gal1-Producing L. lactis Optimized Strain (Measurement of Effector Cytokine Levels)

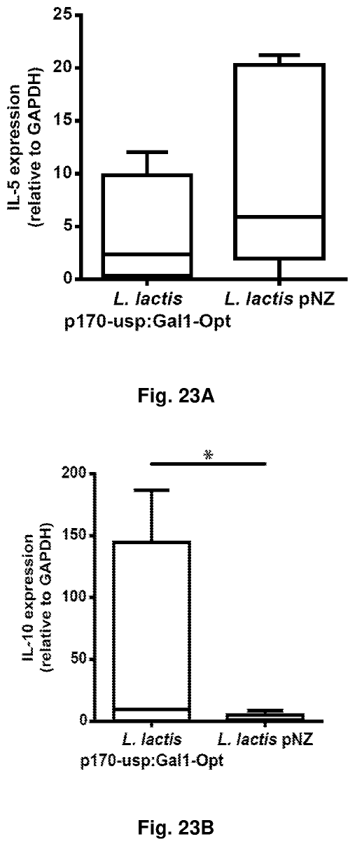

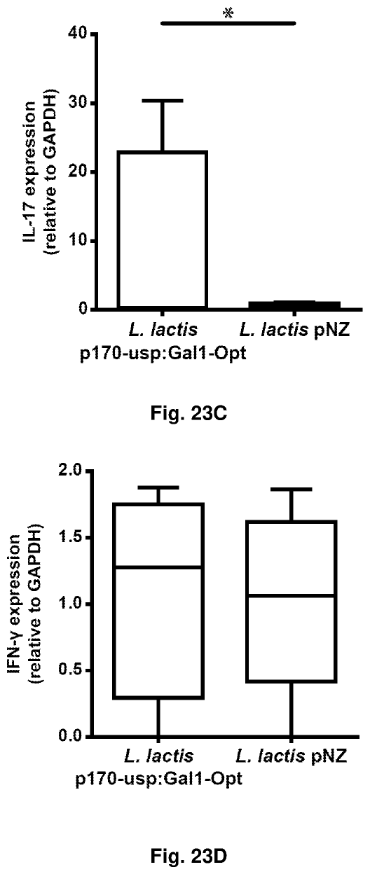

In order to assess the immunomodulatory properties of genetically modified L. lactis, effector cytokine (IL-5, IL-10, IL17, INF-γ) levels in the colon of Lgals1^{-/-} mice coursing intestinal inflammation and treated with L. lactis p170-usp:Gal1-Opt or control strain L. lactis pNZ, were measured (FIG. 23A-23D). There were no changes in the expression of IL-5 (FIG. 23A) or INF-γ (FIG. 23D), but IL-10 (FIG. 23B) and IL-17 (FIG. 23C) were increased in those animals treated with L. lactis p170-usp:Gal1-Opt but not in those receiving the control strain.

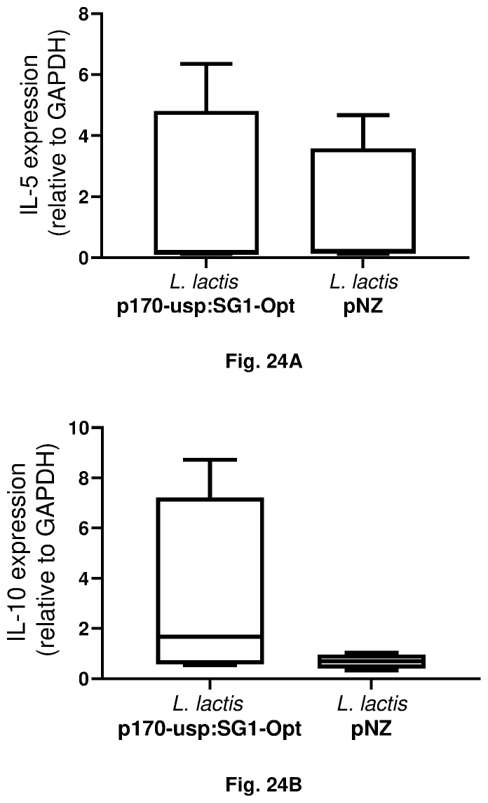

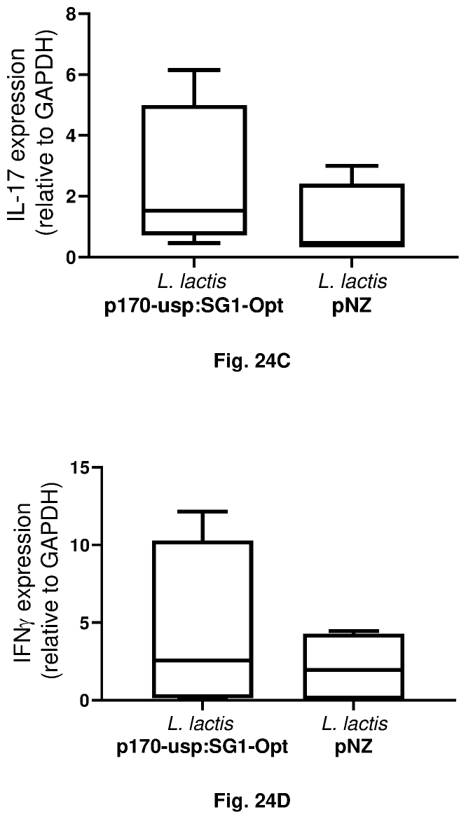

In the case of Lgals1^{-/-} mice daily treated with 1.10⁹ CFU of L. lactis p170-usp:SG1-Opt or control strain (L. lactis pNZ) during TNBS-induced colitis, it may be again observed (FIG. 24A-D) a tendency towards an increase in IL-10 (FIG. 24B) and IL-17 (FIG. 24C) with the SG1-producing strain, similar to what was observed in these animals when received L. lactis p170-usp:Gal1-Opt (FIG. 23B,C). It was previously shown that activated mouse splenocytes from C57Bl/6 mice incubated in vitro with Gal1 and SG1 show both an increase in IL-10, but SG1 induces four times more IL-10 than the WT variant (WO2016172319A1).

These results correlate to previous results by the present inventors showing a similar pattern of IL-5 and IL-10 expression after i.p. administration of rGal1 (100 μg rGal1/per animal, daily) in TNBS-treated Lgals1^{-/-} mice (Morosi et al, 2021). However, and in opposition to said observations after i.p administration of rGal1 in Lgals1^{-/-} mice coursing intestinal inflammation, when animals were treated with L. lactis p170-usp:Gal1-Opt no differences in the levels of IFN-γ were observed (FIG. 23D).

The obtained results suggest that administration of L. lactis p170-usp:Gal1-Opt and L. lactis p170-usp:SG1-Opt by oral gavage increases intestinal IL-10 expression, which could be mediating the anti-inflammatory effects observed in the TNBS experimental model of colitis.

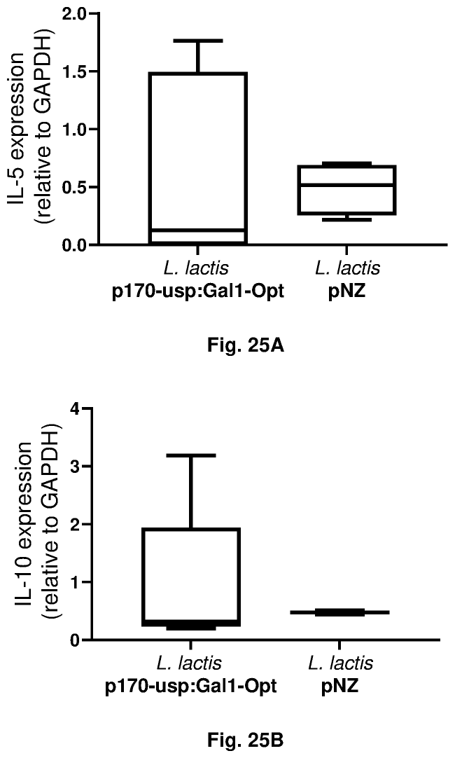

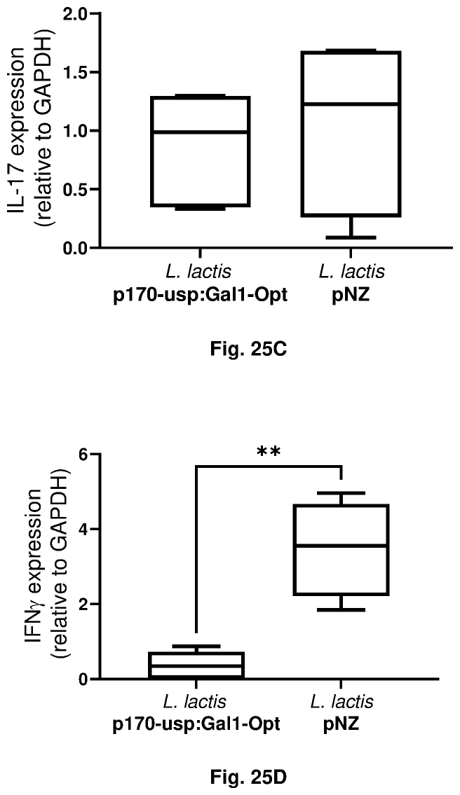

Lastly, measurement of colonic IL-5 (FIG. 25A), IL-10 (FIG. 25B), IL-17 (FIG. 25C) and IFN-γ (FIG. 25D) levels by RT-qPCR in WT mice coursing TNBS-induced intestinal inflammation, and daily treated with 1.10⁹ CFU of L. lactis p170-usp:Gal1-Opt or control strain (L. lactis pNZ) show a significant decrease in a key pro-inflammatory cytokine, IFN-γ, similar to what has been described for i.p. injection of Gal1 in Lgals1^{-/-} mice coursing TNBS-induced colitis (Morosi et al, 2021).

### REFERENCES

Ananthakrishnan, A. N. (2015). Epidemiology and risk factors for IBD. Nat. Rev. Gastroenterol. Hepatol. 12, 205-217.
Bermúdez-Humarán, L. G., Aubry, C., Motta, J. P., Deraison, C., Steidler, L., Vergnolle, N., Chatel, J. M., y Langella, P. (2013). Engineering lactococci and lactobacilli for human health. Curr. Opin. Microbiol. 16, 278-283.
Boyapati, R. K., Kalla, R., Satsangi, J., y Ho, G. T. (2016). Biomarkers in Search of Precision Medicine in IBD. Am. J. Gastroenterol. 111, 1682-1690.
Baumgart, D. C., y Sandborn, W. J. (2012). Crohn's disease. Lancet (London, England) 380, 1590-1605.
Cosnes, J., Gowerrousseau, C., Seksik, P., y Cortot, A. (2011). Epidemiology and natural history of inflammatory bowel diseases. Gastroenterology 140, 1785-1794.
Danese, S., Rudzinski, J., Brandt, W., Dupas, J. L., Peyrin-Biroulet, L., Bouhnik, Y., Kleczkowski, D., Uebel, P., Lukas, M., Knutsson, M., et al. (2014). Tralokinumab (CAT-354), an interleukin 13 antibody, in moderate to severe ulcerative colitis: A phase 2 randomized placebo-controlled study. J. Crohn's Colitis 8, S7-S8.
Duijvestein M, D'Haens G R. Rational and clinical development of the anti-MAdCAM monoclonal antibody for the treatment of IBD. Expert Opin Biol Ther. 2019 April; 19(4):361-366.
Fiorino G, Allocca M, Correale C, Roda G, Furfaro F, Loy L, Zilli A, Peyrin-Biroulet L, Danese S. Positioning ustekinumab in moderate-to-severe ulcerative colitis: new kid on the block. Expert Opin Biol Ther. 2020 April; 20(4):421-427.
Furman D, Campisi J, Verdin E, et al. Chronic inflammation in the etiology of disease across the life span. Nat Med. 2019; 25(12):1822-1832. doi:10.1038/s41591-019-0675-0
Fuss, I. J., Marth, T., Neurath, M. F., Pearlstein, G. R., Jain, A., y Strober, W. (1999). Anti-interleukin 12 treatment regulates apoptosis of Th1 T cells in experimental colitis in mice. Gastroenterology 117, 1078-1088.
Geboes, K. (2003). Histopathology of Crohn's Disease and Ulcerative Colitis. Inflammatory Bowel Diseases. Chapter 18. J. Satsangi, L. R. Sutherland, ed. Churchill Livingstone, London.
Hanson, M. L., Hixon, J. A., Li, W., Felber, B. K., Anver, M. R., Stewart, C. A., Janelsins, B. M., Datta, S. K., Shen, W., McLean, M. H., et al. (2014). Oral delivery of IL-27 recombinant bacteria attenuates immune colitis in mice. Gastroenterology 146, 210-221.
Hazel, K., & O'Connor, A. (2020). Emerging treatments for inflammatory bowel disease. Therapeutic Advances in Chronic Disease, 11, 2040622319899297.
Hohenberger, M., Cardwell, L. A., Oussedik, E., y Feldman, S. R. (2018). Interleukin-17 inhibition: role in psoriasis and inflammatory bowel disease. J. Dermatolog. Treat. 29, 13-18.
Hueber, W., Sands, B. E., Lewitzky, S., Vandemeulebroecke, M., Reinisch, W., Higgins, P. D. R., Wehkamp, J., Feagan, B. G., Yao, M. D., Karczewski, M., et al. (2012). Secukinumab, a human anti-IL-17A monoclonal antibody, for moderate to severe Crohn's disease: Unexpected results of a randomised, double-blind placebo-controlled trial. Gut 61, 1693-1700.
Kamada, N., Seo, S.-U., Chen, G. Y., y Núñez, G. (2013). Role of the gut microbiota in immunity and inflammatory disease. Nat. Rev. Immunol. 13, 321-335.
Madsen S M, Arnau J, Vrang A, Givskov M, Israelsen H. Molecular characterization of the pH-inducible and growth phase-dependent promoter P170 of Lactococcus lactis. Mol Microbiol. 1999 April; 32(1):75-87.
Molodecky, N., Soon, I., Rabi, D., Ghali, W., Ferris, M., Chernoff, G., Benchimol, E., Panaccione, R., Ghosh, S., Barkema, H., et al. (2012). Increasing incidence and prevalence of the inflammatory bowel diseases with time, based on systematic review. Gastroenterology. 142, 46-54.
Morosi L G, Cutine A M, Cagnoni A J, Manselle-Cocco M N, Croci D O, Merlo J P, Morales R M, May M, Pérez-Sáez J M, Girotti M R, Méndez-Huergo S P, Pucci B, Gil A H, Huernos S P, Docena G H, Sambuelli A M, Toscano M A, Rabinovich G A, Mariño K V. Control of intestinal inflammation by glycosylation-dependent lectin-driven immunoregulatory circuits. Sci Adv. 2021-6-18; 7(25):eabf8630. doi:10.1126/sciadv.abf8630.
Muglia, C., Mercer, N., Toscano, M. A., Schattner, M., Pozner, R., Cerliani, J. P., Gobbi, R. P., Rabinovich, G. A., y Docena, G. H. (2011). The glycan-binding protein galectin-1 controls survival of epithelial cells along the crypt-villus axis of small intestine. Cell Death Dis. 2, e163.
Muglia, C. I., Gobbi, R. P., Smaldini, P., Delgado, M. L. O., Candia, M., Zanuzzi, C., Sambuelli, A., Rocca, A., Toscano, M. A., Rabinovich, G. A., et al. (2016). Inflammation Controls Sensitivity of Human and Mouse Intestinal Epithelial Cells to Galectin-1. J. Cell. Physiol. 231, 1575-1585.
Nio-Kobayashi, J., Takahashi-Iwanaga, H., y Iwanaga, T. (2009). Immunohistochemical localization of six galectin subtypes in the mouse digestive tract. J. Histochem. Cytochem. 57, 41-50.
Neurath, M. F. (2014). Cytokines in inflammatory bowel disease. Nat. Rev. Immunol. 14, 329-342.
Ordás, I., Eckmann, L., Talamini, M., Baumgart, D. C., y Sandborn, W. J. (2012). Ulcerative colitis. Lancet (London, England) 380, 1606-1619.
Pace, K. E., Hahn, H. P., y Baum, L. G. (2003). Preparation of recombinant human Galectin-1 and use in T-cell death assays. MethodsEnzymol. 363, 499-518.
Papa Gobbi, R., De Francesco, N., Bondar, C., Muglia, C., Chirdo, F., Rumbo, M., Rocca, A., Toscano, M. A., Sambuelli, A., Rabinovich, G. A., et al. (2016). A galectin-specific signature in the gut delineates Crohn's disease and ulcerative colitis from other human inflammatory intestinal disorders. BioFactors 42, 93-105.
Paclik D, Lohse K, Wiedenmann B, Dignass A U, Sturm A. Galectin-2 and -4, but not galectin-1, promote intestinal epithelial wound healing in vitro through a TGF-beta-independent mechanism. Inflamm Bowel Dis. 2008 October; 14(10):1366-72.
Rabinovich, G. A; Di Lella, S.; Estrin, D. and Caramelo, J. Gal-1 variants having immunomodulating properties and methods of using the same. WO2016172319A1.
Reinisch, W., de Villiers, W., Bene, L., Simon, L., Racz, I., Katz, S., Altorjay, I., Feagan, B., Riff, D., Bernstein, C. N., et al. (2010). Fontolizumab in moderate to severe Crohn's disease: a phase 2, randomized, double-blind, placebo-controlled, multiple-dose study. Inflamm. Bowel Dis. 16, 233-242.
Rosenblum, M. D., Gratz, I. K., Paw, J. S., y Abbas, A. K. (2012). Treating human autoimmunity: Current practice and future prospects. Sci. Transl. Med. 4.
Santucci L, Fiorucci S, Rubinstein N, Mencarelli A, Palazzetti B, Federici B, Rabinovich G A, Morelli A. (2003) Galectin-1 suppresses experimental colitis in mice. Gastroenterology 124(5), 1381-94.
Sommer K, Wiendl M, Müller T M, Heidbreder K, Voskens C, Neurath M F, Zundler S. Intestinal Mucosal Wound Healing and Barrier Integrity in IBD-Crosstalk and Trafficking of Cellular Players. Front Med (Lausanne). 2021-3-23; 8:643973.
Song, A. A. L., In, L. L. A., Lim, S. H. E., y Rahim, R. A. (2017). A review on Lactococcus lactis: From food to factory. Microb. Cell Fact. 16, 55.
Starossom, S. C., Mascanfroni, I. D., Imitola, J., Cao, L., Raddassi, K., Hernandez, S. F., Bassil, R., Croci, D. O., Cerliani, J. P., Delacour, D., et al. (2012). Galectin-1 deactivates classically activated microglia and protects from inflammation-induced neurodegeneration. Immunity 37, 249-263.
Steidler, L., Hans, W., Schotte, L., Neirynck, S., Obermeier, F., Falk, W., Fiers, W., y Remaut, E. (2000). Treatment of murine colitis by Lactococcus lactis secreting interleukin-10. Science 289, 1352-1355.
Suarez-Arnedo, A., Torres Figueroa, F., Clavijo, C., Arbeláez, P., Cruz, J. C., & Muñoz-Camargo, C. (2020). An image J plugin for the high throughput image analysis of in vitro scratch wound healing assays. PloS one, 15(7), e0232565.
Sundblad, V., Morosi, L. G., Geffner, J. R., y Rabinovich, G. A. (2017). Galectin-1: A jack-of-all-trades in the resolution of acute and chronic inflammation. J. Immunol. 199, 3721-3730.
Sýkora J, Pomahačová R, Kreslová M, Cvalínová D, Štych P, Schwarz J. Current global trends in the incidence of pediatric-onset inflammatory bowel disease. World J Gastroenterol. 2018; 24(25):2741-2763.
Toscano M. A., Martínez Allo V. C., Cutine A. M., Rabinovich G. A., Mariño K. V. (2018) Untangling galectin-driven regulatory circuits in autoimmune inflammation. Trends in Molecular Medicine 24(4):348-363.
Wirtz, S., Popp, V., Kindermann, M., Gerlach, K., Weigmann, B., Fichtner-Feigl, S., Neurath, M. F. (2017). Chemically induced mouse models of acute and chronic intestinal inflammation. Nat. Protoc. 12, 1295-1309.

### SEQUENCE LISTING

<110> CONICET
<120> OPTIMIZED GALECTIN-1 FOR THERAPEUTIC CONTROL OF INTESTINAL INFLAMMATION
<130> 00-conicet
<160> 21
<170> PatentIn version 3.5
<210> 1
<211> 408
<212> DNA
<213> human Gal1
<400> 1
atggcttgtggtcttgttgctagtaatcttaatttgaaacctggtgaatgtttgcgagtt 60
cgtggtgaagttgctcctgatgctaaatcatttgttcttaatcttggtaaagattcaaat 120
aatttgtgtcttcattttaatccacgttttaatgctcatggagatgcaaatacaattgtt 180
tgtaattcaaaagatggtggagcttggggtactgaacaaagagaagcagtatttccattt 240
caacctggatctgttgctgaagtatgtattacatttgatcaagcaaatcttactgtaaaa 300
cttccagatggttatgaatttaaatttcctaatcgacttaatttggaagctattaattat 360
atggctgctgatggagattttaaaattaaatgtgttgcttttgattaa 408
<210> 2
<211> 422
<212> DNA
<213> Artificial Sequence
<220>
<223> SuperGal1 with optimized sequence and including secretion peptide usp45
<400> 2
ccatggcttcaggtcttgttgctagtaatcttaatttgaaacctggtgaatcattgcgag 60
ttcgtggtgaagttgctcctgatgctaaatcatttgttcttaatcttggtaaagattcaa 120
ataatttgtgtcttcattttaatccacgttttaatgctaatggagatgcaaatacaattg 180
tttgtaattcaaaagatggtggagcttggggtactgaacaaagagaagcagtatttccat 240
ttcaacctggatctgttgctgaagtatgtattacatttgatcaagcaaatcttactgtaa 300
aacttccagatggttatgaatttaaatttcctaatcgacttaatttggaagctattaatt 360
atatggctgctgatggagattttaaaattaaatgtgttgcttttgattaaggtaccaagc 420
tt 422
<210> 3
<211> 422
<212> DNA
<213> Artificial Sequence
<220>
<223> SuperGal2 with optimized sequence and including secretion peptide usp45
<400> 3
ccatggcttcaggtcttgttgctagtaatcttaatttgaaacctggtgaatcattgcgag 60
ttcgtggtgaagttgctcctgatgctaaatcatttgttcttaatcttggtaaagattcaa 120
ataatttgtgtcttcattttaatccacgttttaatgcttatggagatgcaaatacaattg 180
tttgtaattcaaaagatggtggagcttggggtactgaacaaagagaagcagtatttccat 240
ttcaacctggatctgttgctgaagtatgtattacatttgatcaagcaaatcttactgtaa 300
aacttccagatggttatgaatttaaatttcctaatcgacttaatttggaagctattaatt 360
atatggctgctgatggagattttaaaattaaatgtgttgcttttgattaaggtaccaagc 420
tt 422
<210> 4
<211> 422
<212> DNA
<213> Artificial Sequence
<220>
<223> SuperGal3 with optimized sequence and including secretion peptide usp45
<400> 4
ccatggcttcaggtcttgttgctagtaatcttaatttgaaacctggtgaatgtttgcgag 60
ttcgtggtgaagttgctcctgatgctaaatcatttgttcttaatcttggtaaagattcaa 120
ataatttgtgtcttcattttaatccacgttttaatgctaatggagatgcaaatacaattg 180
tttgtaattcaaaagatggtggagcttggggtactgaacaaagagaagcagtatttccat 240
ttcaacctggatctgttgctgaagtatcaattacatttgatcaagcaaatcttactgtaa 300
aacttccagatggttatgaatttaaatttcctaatcgacttaatttggaagctattaatt 360
atatggctgctgatggagattttaaaattaaatgtgttgcttttgattaaggtaccaagc 420
tt 422
<210> 5
<211> 422
<212> DNA
<213> Artificial Sequence
<220>
<223> SuperGal4 with optimized sequence and including secretion peptide usp45
<400> 5
ccatggcttcaggtcttgttgctagtaatcttaatttgaaacctggtgaatgtttgcgag 60
ttcgtggtgaagttgctcctgatgctaaatcatttgttcttaatcttggtaaagattcaa 120
ataatttgtgtcttcattttaatccacgttttaatgcttatggagatgcaaatacaattg 180
tttgtaattcaaaagatggtggagcttggggtactgaacaaagagaagcagtatttccat 240
ttcaacctggatctgttgctgaagtatcaattacatttgatcaagcaaatcttactgtaa 300
aacttccagatggttatgaatttaaatttcctaatcgacttaatttggaagctattaatt 360
atatggctgctgatggagattttaaaattaaatgtgttgcttttgattaaggtaccaagc 420
tt 422
<210> 6
<211> 528
<212> DNA
<213> Homo sapiens
<400> 6
atctctctcgggtggagtcttctgacagctggtgcgcctgcccgggaacatcctcctgga 60
ctcaatcatggcttgtggtctggtcgccagcaacctgaatctcaaacctggagagtgcct 120
tcgagtgcgaggcgaggtggctcctgacgctaagagcttcgtgctgaacctgggcaaaga 180
cagcaacaacctgtgcctgcacttcaaccctcgcttcaacgcccacggcgacgccaacac 240
catcgtgtgcaacagcaaggacggcggggcctgggggaccgagcagcgggaggctgtctt 300
tcccttccagcctggaagtgttgcagaggtgtgcatcaccttcgaccaggccaacctgac 360
cgtcaagctgccagatggatacgaattcaagttccccaaccgcctcaacctggaggccat 420
caactacatggcagctgacggtgacttcaagatcaaatgtgtggcctttgactgaaatca 480
gccagcccatggcccccaataaaggcagctgcctctgctccctctgaa 528
<210> 7
<211> 20
<212> DNA
<213> Artificial Sequence
<220>
<223> Gal1 forward primer
<400> 7
tcagcctggt caaaggtgat 20
<210> 8
<211> 20
<212> DNA
<213> Artificial Sequence
<220>
<223> Gal1 reverse primer
<400> 8
tgaacctgggaaaagacagc 20
<210> 9
<211> 21
<212> DNA
<213> Artificial Sequence
<220>
<223> IFN forward primer
<400> 9
ggctgttactgccacggcac a 21
<210> 10
<211> 25
<212> DNA
<213> Artificial Sequence
<220>
<223> IFN reverse primer
<400> 10
caccatccttttgccagttcctcca 25
<210> 11
<211> 20
<212> DNA
<213> Artificial Sequence
<220>
<223> IL5 forward primer
<400> 11
acacagctgt ccgctcaccg 20
<210> 12
<211> 21
<212> DNA
<213> Artificial Sequence
<220>
<223> IL5 reverse primer
<400> 12
tccacagtacccccacggac a 21
<210> 13
<211> 20
<212> DNA
<213> Artificial Sequence
<220>
<223> IL10 forward primer
<400> 13
ttccctgggt gagaagctga 20
<210> 14
<211> 20
<212> DNA
<213> Artificial Sequence
<220>
<223> IL10 reverse primer
<400> 14
cttcacctgc tccactgcct 20
<210> 15
<211> 22
<212> DNA
<213> Artificial Sequence
<220>
<223> IL17 forward primer
<400> 15
ctccagaaggccctcagact ac 22
<210> 16
<211> 24
<212> DNA
<213> Artificial Sequence
<220>
<223> IL17 reverse primer
<400> 16
agctttccct ccgcattgac acag 24
<210> 17
<211> 19
<212> DNA
<213> Artificial Sequence
<220>
<223> GADPH forward primer
<400> 17
cagaacatca tccctgcat 19
<210> 18
<211> 21
<212> DNA
<213> Artificial Sequence
<220>
<223> GADPH reverse primer
<400> 18
gttcagctctgggatgacct t 21
<210> 19
<211> 239
<212> DNA
<213> Artificial Sequence
<220>
<223> p170 promoter sequence
<400> 19
aagcttagatctggaagtaattctagagctttgcatgtctataaaaaaatgggattccaa 60
attaccatatcaatatgcgaaaagaactatgaatatccactccatttttggttgccatt 120
tgttaacgctgcctcctctccctagtgctataataaaaatggcccattttggaacagact 180
tctactattttgttgtctagtagcgggtctaaattagggtactgcagaaggaggtattc 239
<210> 20
<211> 84
<212> DNA
<213> Artificial Sequence
<220>
<223> Usp45 secretion signal peptide sequence
<400> 20
atgaaaaaaaagattatctcagctattttaatgtctacagtgatactttcagcagcagcc 60
ccgttgtcaggtgtttacgctgcc 84
<210> 21
<211> 18
<212> DNA
<213> Artificial Sequence
<220>
<223> spacer sequence
<400> 21
cgggtctaaa ttagggta 18

1. A nucleotide construct comprising a promoter sequence and an insert, wherein
said insert comprises a galectin-1 nucleotide sequence fused to a signal secretion peptide, wherein the galectin-1 nucleotide sequence is selected from the group consisting of SEQ ID NO: 1-5, and wherein said secretion peptide is usp45 of SEQ ID NO: 20; and
said promoter sequence has a sequence as depicted in SEQ ID NO: 19.

2. The nucleotide construct according to claim 1, wherein the galectin-1 nucleotide sequence has a nucleotide sequence as depicted in SEQ ID NO: 1.

3. The nucleotide construct according to claim 1, wherein the galectin-1 nucleotide sequence has a nucleotide sequence as depicted in SEQ ID NO: 2.

4. The nucleotide construct according to claim 1, wherein the galectin-1 nucleotide sequence has a nucleotide sequence as depicted in SEQ ID NO: 3.

5. The nucleotide construct according to claim 1, wherein the galectin-1 nucleotide sequence has a nucleotide sequence as depicted in SEQ ID NO: 4.

6. The nucleotide construct according to claim 1, wherein the galectin-1 nucleotide sequence has a nucleotide sequence as depicted in SEQ ID NO: 5.

7.-11. (canceled)

12. A transformed Lactococcus lactis strain selected from the group consisting of:
a transformed Lactococcus lactis strain expressing Galectin-1 WT, which is deposited under Accession Number RGM3045;
a transformed Lactococcus lactis strain expressing Gal1 variant SG1, which is deposited under Accession Number RGM3046;
a transformed Lactococcus lactis strain expressing Gal1 variant SG2, which is deposited under Accession Number RGM3047;
a transformed Lactococcus lactis strain expressing Gal1 variant SG3, which is deposited under Accession Number RGM3048; and
a transformed Lactococcus lactis strain expressing Gal1 variant SG4, which is deposited under Accession Number RGM3049.

13.-17. (canceled)

18. A method for controlling inflammation in a subject suffering from an inflammation-mediated disease, wherein the inflammation-mediated disease is an inflammatory bowel disease (IBD), or an immune-mediated inflammatory disease, wherein the immune-mediated inflammatory disease is selected from rheumatoid arthritis and multiple sclerosis, the method comprising orally administering to said subject a transformed Lactococcus lactis strain according to claim 12.

19. The method according to claim 18, wherein the IBD is selected from ulcerative colitis and Crohn's disease.

20. (canceled)

21. The method according to claim 18, wherein the transformed Lactococcus lactis strain is administered in combination with at least one additional therapeutic agent.

22. The method according to claim 21, wherein the at least one therapeutic agent includes at least one immunosuppressive drug.

23. The method according to claim 21, wherein the co-administration of at least one additional therapeutic agent is sequential or simultaneous.

24.-33. (canceled)
